(19) United States
(12) Patent Application Publication
(10) Pub. No.: US 2003/0036079 A1
(43) Pub. Date: Feb. 20, 2003

(54) Gene expression alterations underlying the retardation of aging by caloric restriction in mammals

(76) Inventors: Richard H. Weindruch, Madison, WI (US); Tomas A. Prolla, Madison, WI (US); Cheol-Koo Lee, Madison, WI (US); Tsuyoshi Kayo, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/178,296

(22) Filed: Jun. 24, 2002

Related U.S. Application Data
(60) Provisional application No. 60/300,949, filed on Jun. 26, 2001.

(51) Int. Cl. C12Q 1/68; G01N 33/567

(52) U.S. Cl. 435/6; 435/7.21

(57) ABSTRACT

A method of measuring the relative metabolic state of a multicellular organism is disclosed. In one embodiment, the method comprises the steps of: (a) obtaining a sample of nucleic acid isolated from the organism's organ, tissue or cell, wherein the nucleic acid is RNA or a cDNA copy of RNA, (b) determining the gene expression pattern of at least one of the genes selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088 and (c) determining whether the gene expression profile of step (b) is more similar to a CR-induced metabolic state or a standard diet metabolic state.

### CROSS-REFERENCE TO RELATED APPLICATION

This application claims priority to 60/300,949, filed Jun. 26, 2001 and incorporated by reference herein.

### STATEMENT REGARDING FEDERALLY SPONSORED RESEARCH OR DEVELOPMENT

This invention was made with United States government support awarded by the following agencies: NIH CA78723. The United States has certain rights in this invention.

### BACKGROUND OF THE INVENTION

A common feature of most multicellular organisms is the progressive and irreversible physiological decline that characterizes senescence. Although genetic and environmental factors can influence the aging process, the molecular basis of senescence remains unknown. Postulated mechanisms include cumulative damage to DNA leading to genomic instability, epigenetic alterations that lead to altered gene expression patterns, telomere shortening in replicative cells, oxidative damage to critical macromolecules and nonenzymatic glycation of long-lived proteins (Jazwinski, 1996; Martin, et al., 1996; Johnson, et al., 1999; Beckman and Ames, 1998). Factors which contribute to the difficulty of elucidating mechanisms and testing interventions include the complexity of organismal senescence and the lack of molecular markers of biological age (so-called biomarkers of aging). Aging is complex in that underlying mechanisms in tissues with limited regenerative capacities (e.g., skeletal and cardiac muscle, brain), which are composed mainly of postmitotic (non-dividing) cells, may differ markedly from those operative in proliferative tissues. Accordingly, approaches which provide a global assessment of senescence in specific tissues would greatly increase understanding of the aging process and the possibility of pharmaceutical, genetic or nutritional intervention.

Genetic manipulation of the aging process in multicellular organisms has been achieved in Drosophila, through the over-expression of catalase and Cu/Zn superoxide dismutase (Orr and Sohal, 1994; Parkes, et al., 1998), in the nematode C. elegans, through alterations in the insulin receptor signaling pathway (Ogg, et al., 1997; Paradis and Ruvkun, 1998; Tissenbaum and Ruvkun, 1998), and through the selection of stress-resistant mutants in either organism (Johnson, 1990; Murakami and Johnson, 1996; Lin, et al., 1998). In mammals, there has been limited success in the identification of genes that control aging rates. Mutations in the Werner Syndrome locus (WRN) accelerate the onset of a subset of aging-related pathologies in humans, but the role of the WRN gene product in the modulation of normal aging is unknown (Yu, et al.,1996; Lombard and Guarente, 1996).

In contrast to the current lack of genetic interventions to retard the aging process in mammals, caloric restriction (CR) appears to slow the intrinsic rate of aging (Weindruch and Walford, 1988; Fishbein, 1991, Yu, 1994). Most studies have involved laboratory rodents which, when subjected to a long-term, 25-50% reduction in calorie intake without essential nutrient deficiency, display delayed onset of age-associated pathological and physiological changes and extension of maximum lifespan.

The effects of CR on average and maximum lifespan and mortality rate parameters in rodents as well as on age-associated pathological and physiological changes strongly support the view that CR slows fundamental aspects of the aging process (reviewed by Weindruch and Walford, 1988). This hypothesis is also supported by the fact that CR can retard the aging process in diverse species, such as Tokophyra (a protozoan), Daphnia (the water flea) and Lebistes (the guppy). Despite intensive investigation, the mechanism(s) of aging retardation by CR remains unknown. In part, this derives from the observation that animals on CR display physiological changes that support many current aging theories. Indeed, CR reduces not only O₂ consumption on a whole-animal basis, but also thyroid hormone levels and body temperature, suggesting a lower metabolic rate. CR also reduces blood glucose levels, increases insulin sensitivity and preserves certain age-sensitive immunological functions.

A theory that is gaining favor is that CR exerts its mechanism of action through the induction of a global metabolic response that results in higher metabolic efficiency, lower production of toxic byproducts of metabolism, and the induction of specific stress adaptation responses (McCarter, 1995; Sohal and Weindruch, 1996; Frame, et al., 1998; Masoro, 1998). Global stress adaptations, such as that mediated by the oxyR regulon, have been well characterized in bacteria (Pomposiello and Demple, 2001), and likely exist in mammals. Evidence linking metabolic control to aging derives from work in C. elegans, which demonstrates that mutations in the insulin-related transcription factor DAF-16 control lifespan (Ogg, et al., 1997). Interestingly, mutations in DAF-2, another gene involved in metabolic control, are also associated with elevated resistance to thermal exposure and oxidative stress (Honda and Honda, 1999). Identification of the genes that mediate the effects of CR on metabolic response would allow for the development of pharmaceutical compounds or genetic interventions that mimic the effects of CR, leading to improved health and disease prevention.

Recent studies also suggest that CR has a beneficial effect in experimental models of neurodegeneration. The vulnerability of midbrain dopaminergic neurons to MPTP toxicity is decreased, and motor function improved, in mice maintained on CR (Duan and Mattson, 1999). An animal model of Huntington's Disease involves administration of the succinate dehydrogenase inhibitor 3-nitropropionic acid (3NP) to rats. Maintenance of rats on a CR regimen for several months prior to administration of 3NP results in increased resistance of striatal neurons to 3NP and improved motor function (Bruce-Keller, et al., 1999). Emerging findings from studies of human populations also support a protective effect of CR against age-related neurodegenerative disorders. Studies of a large cohort of people living in New York City have revealed that individuals with the lowest daily calorie intakes have the lowest risk for Alzheimer's disease (Mayeux, et al., 1999) and Parkinson's disease (Logroscino, et al., 1996). Moreover, it was recently shown that the incidence of Alzheimer's disease is decreased by more than 50% when genetically similar populations of blacks live in communities where they consume a reduced-calorie diet (Hendrie, et al., 2001). Therefore, identification of the genes that mediate the effects of CR on the central nervous system may provide targets for the development of strategies to prevent or retard age-associated neurodegenerative diseases.

Because CR is likely to affect many metabolic pathways, approaches which provide a global assessment of the influences of CR in multiple tissues would greatly increase our understanding of how this dietary regimen retards aging and prevents diseases. Furthermore, the identification of specific genes which are altered in expression by CR in multiple tissues would result in the discovery of targets for the development of pharmaceutical compounds that mimic the metabolic effects of this dietary regimen. Additionally, such genes represent biomarkers of the metabolic state induced by CR and, therefore, can be used in screening assays for the identification of lead compounds that mimic the effects of CR at the gene expression and metabolic levels.

### SUMMARY OF THE INVENTION

In one embodiment, the present invention is a method of measuring a relative metabolic state of a multicellular organism comprising the steps of: (a) obtaining a sample from a subject; (b) determining the gene expression pattern of at least one of the genes selected from the group consisting of ORFs D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088; and (c) determining whether the gene expression profile of step (b) is more similar to a CR-induced metabolic state or a standard diet metabolic state.

In another embodiment, the present invention is a method for screening a compound for the ability to modulate the metabolic state in a multicellular organism comprising the steps of: (a) dividing test organisms into first and second groups; (b) exposing the organisms of the first group to a test compound; (c) analyzing samples of the first and second groups for the gene expression pattern of at least one of the genes selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088; and (d) comparing the analysis of the first and second groups and identifying test compounds that modify the expression of the sequences of step (c) in the first group such that the expression patterns are more similar to those observed in CR-treated animals.

Other embodiments of the invention are described below.

### BRIEF DESCRIPTION OF THE SEVERAL VIEWS OF THE DRAWINGS

FIGS. 1-11 are individual bar graphs disclosing the full change of mRNAs and lines showing signal intensities corresponding to individual sequences in tissues from caloric-restricted and normally-fed mice.

FIG. 1A-C discloses fold changes in gene expression of genes upregulated by CR in all six tissues (cerebellum, neocortex, gastrocnemius, heart, kidney and liver). FIG. 1A discloses changes in R74626. FIG. 1B discloses changes in U79163. FIG. 1C discloses changes in D31966.

FIG. 2A-E discloses fold changes in gene expression of genes down-regulated by CR in all six tissues. FIG. 2A discloses changes in U79523. FIG. 2B discloses changes in M22531. FIG. 2C discloses changes in U43285. FIG. 2D discloses changes in X81059. FIG. 2E discloses changes in X84239.

FIG. 3A-D discloses fold changes in gene expression in genes upregulated by CR in all but gastrocnemius. FIG. 3A discloses changes in U84411. FIG. 3B discloses changes in M70642. FIG. 3C discloses changes in U37775. FIG. 3D discloses changes in D38117.

FIG. 4A-C discloses fold changes in gene expression of genes upregulated by CR in all tissues but heart. FIG. 4A discloses changes in D87117. FIG. 4B discloses changes in U51167. FIG. 4C discloses changes in U31966.

FIG. 5A-E discloses fold changes in gene expression of genes upregulated by CR in all tissues but kidney. FIG. 5A discloses changes in M97900. FIG. 5B discloses changes in U43836. FIG. 5C discloses changes in U32684. FIG. 5D discloses changes in U60001. FIG. 5E discloses changes in X61450.

FIG. 6A-E discloses fold changes in gene expression of genes upregulated by CR in all tissues but liver. FIG. 6A discloses changes in L08651. FIG. 6B discloses changes in U28917. FIG. 6C discloses changes in U49507. FIG. 6D discloses changes in X59846. FIG. 6E discloses changes in D49473.

FIG. 7 discloses fold changes in gene expression of a gene downregulated by CR in all tissues but gastrocnemius. FIG. 7 discloses changes in X00958.

FIG. 8A-B discloses fold changes in gene expression of genes downregulated by CR in all tissues but heart. FIG. 8A discloses changes in K03235. FIG. 8B discloses changes in Z48238.

FIG. 9 discloses fold changes in gene expression of a gene downregulated by CR in all tissues but kidney. FIG. 9 discloses changes in M60596.

FIG. 10A-DD discloses fold changes in gene expression of genes upregulated by CR in all four post-mitotic tissues. FIG. 10A discloses changes in AA117417. FIG. 10B discloses changes in AF007267. FIG. 10C discloses changes in AF011644. FIG. 10D discloses changes in AJ001101. FIG. 10E discloses changes in C79471. FIG. 10F discloses changes in D16333. FIG. 10G discloses changes in D49744. FIG. 10H discloses changes in D83146. FIG. 10I discloses changes in L29123. FIG. 10J discloses changes in D86424. FIG. 10K discloses changes in L40632. FIG. 10L discloses changes in M74555. FIG. 10M discloses changes in M91380. FIG. 10N discloses changes in M93428. FIG. 10O discloses changes in U19799. FIG. 10P discloses changes in U20344. FIG. 10Q discloses changes in U34973. FIG. 10R discloses changes in U35312. FIG. 10S discloses changes in U35646. FIG. 10T discloses changes in U43512. FIG. 10U discloses changes in U47008. FIG. 10V discloses changes in U47543. FIG. 10W discloses changes in U56773. FIG. 10X discloses changes in X06407. FIG. 10Y discloses changes in X54352. FIG. 10Z discloses changes in X84037. FIG. 10AA discloses changes in Y00746. FIG. 10BB discloses changes in Y07688. FIG. 10CC discloses changes in Z19581. FIG. 10DD discloses changes in Z46966.

FIG. 11A-Y discloses fold changes of gene expression of genes downregulated by CR in four post-mitotic tissues. FIG. 11A discloses changes in AF003695. FIG. 11B discloses changes in AF020772. FIG. 11C discloses changes in C76063. FIG. 11D discloses changes in C79663. FIG. 11E discloses changes in D86344. FIG. 11F discloses changes in D67076. FIG. 11G discloses changes in D10715. FIG. 11H discloses changes in D12713. FIG. 11I discloses changes in L10244. FIG. 11J discloses changes in L18888. FIG. 11K discloses changes in M57966. FIG. 11L discloses changes in M58564. FIG. 11M discloses changes in U19463. FIG. 11N discloses changes in U25844. FIG. 11O discloses changes in U27830. FIG. 11P discloses changes in U35623. FIG. 11Q discloses changes in U43892. FIG. 11R discloses changes in U51204. FIG. 11S discloses changes in U75321. FIG. 11T discloses changes in U84207. FIG. 11U discloses changes in X52914. FIG. 11V discloses changes in X54424. FIG. 11W discloses changes in X75926. FIG. 11X discloses changes in X99921. FIG. 11Y discloses changes in Z47088.

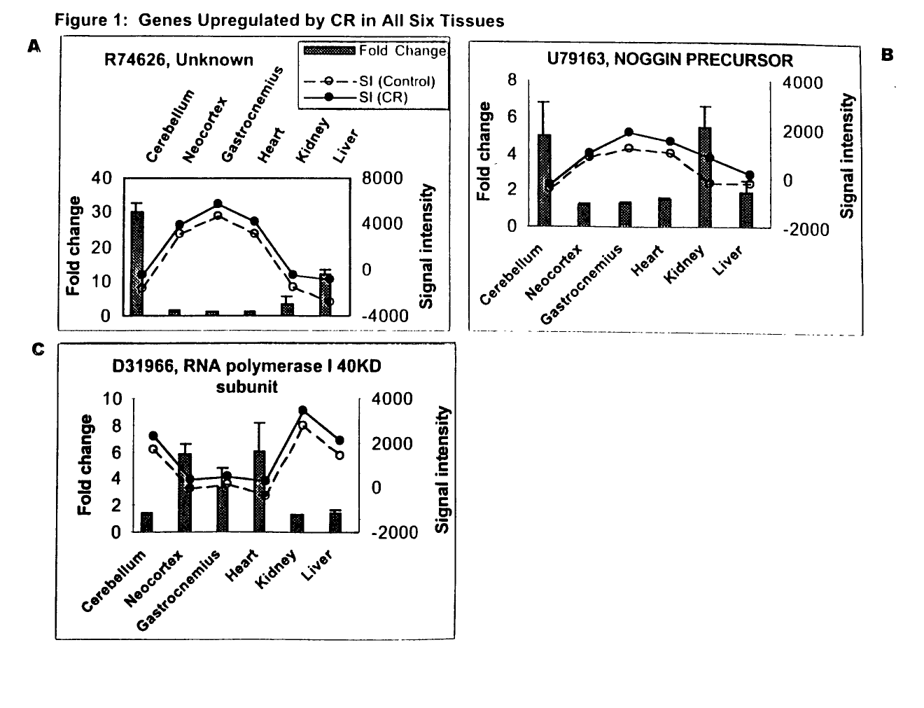

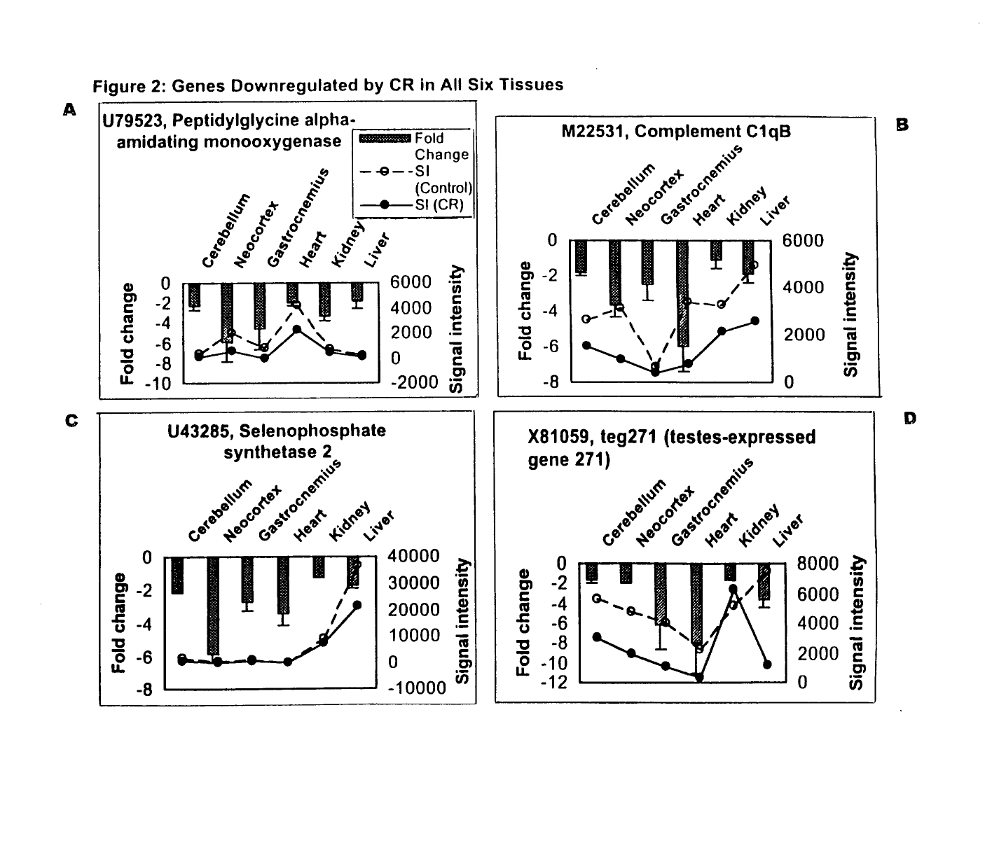

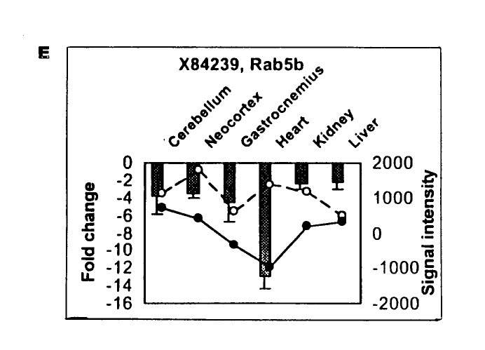

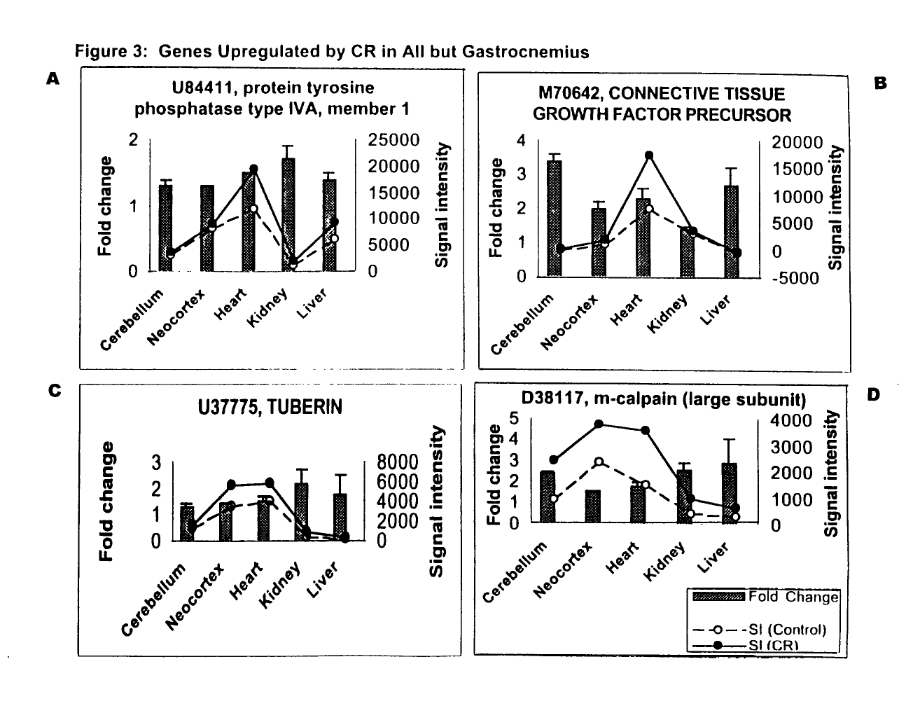

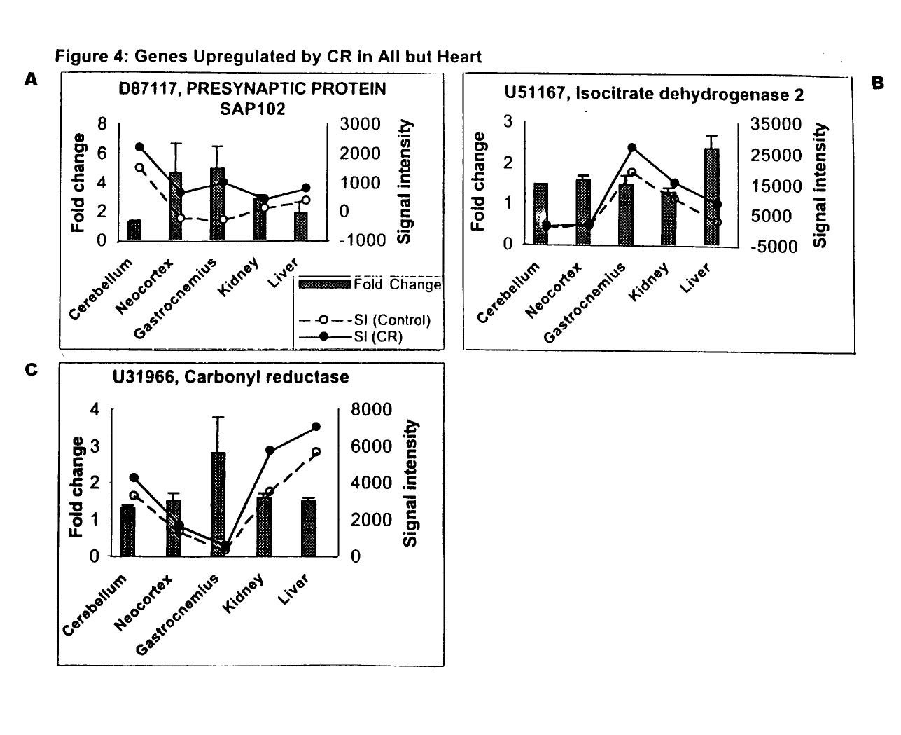

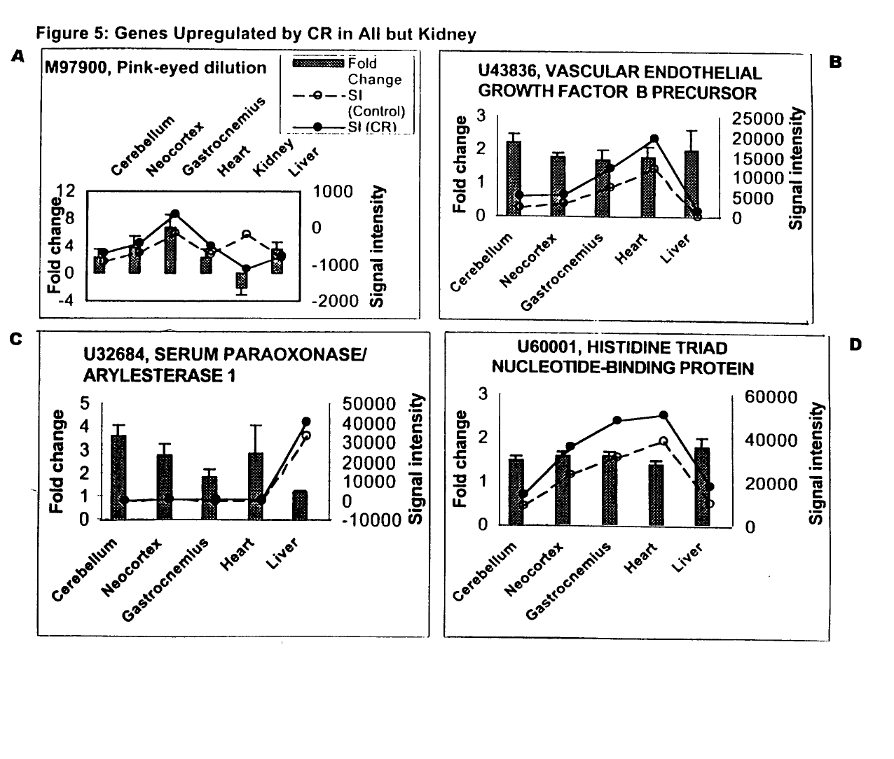

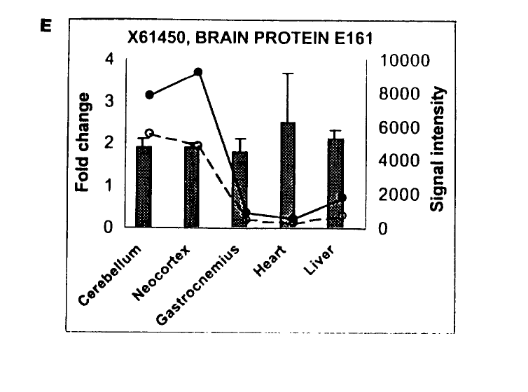

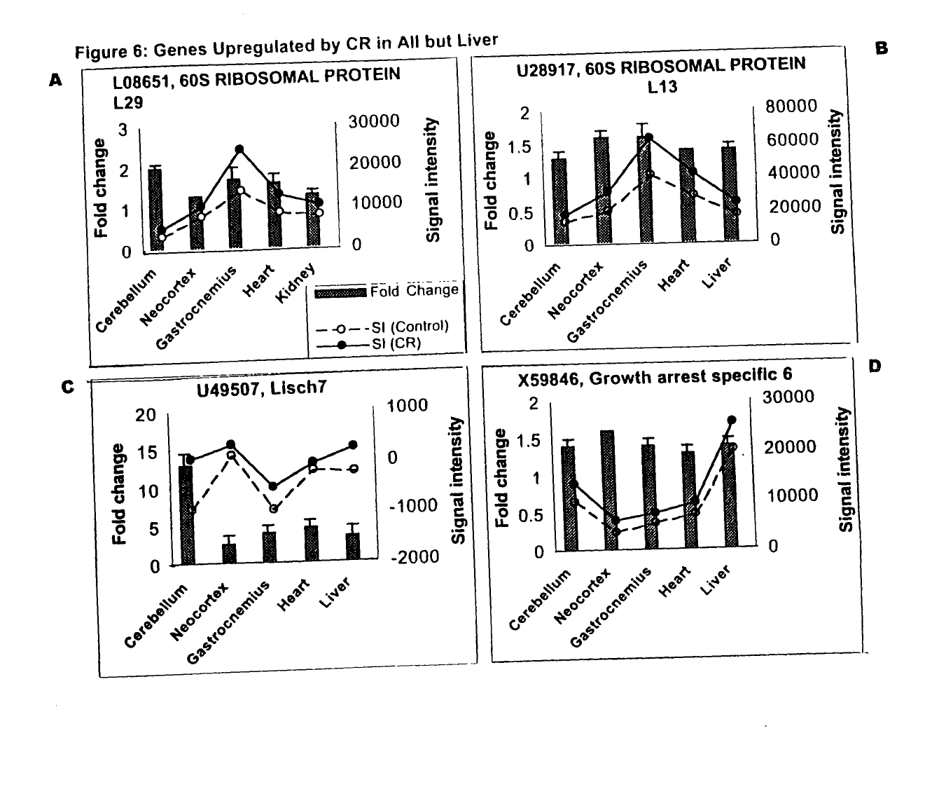

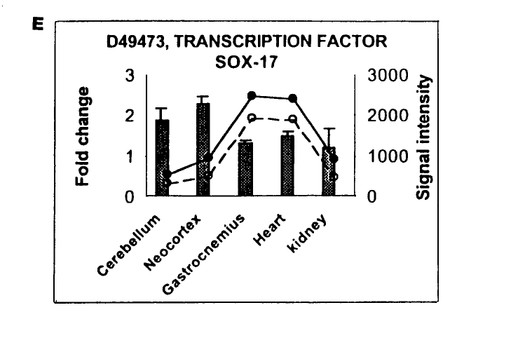

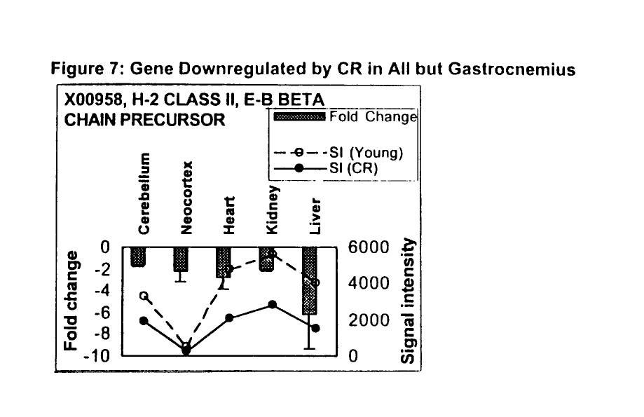

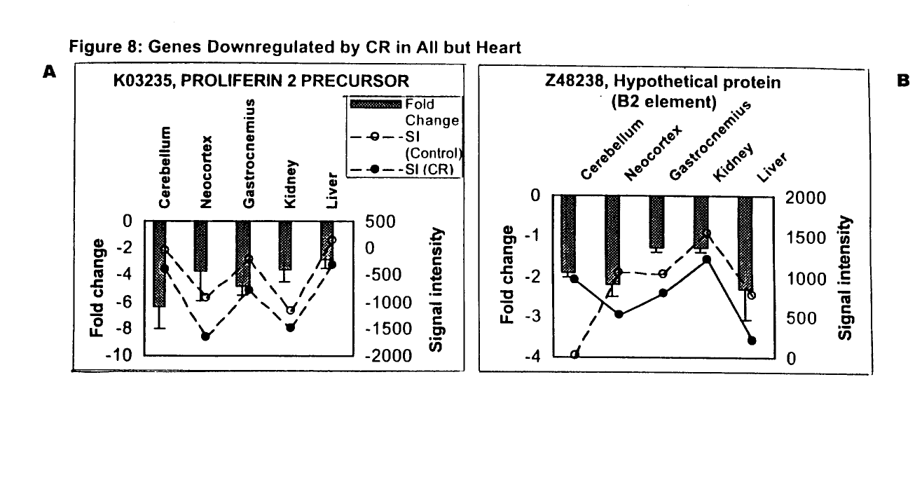

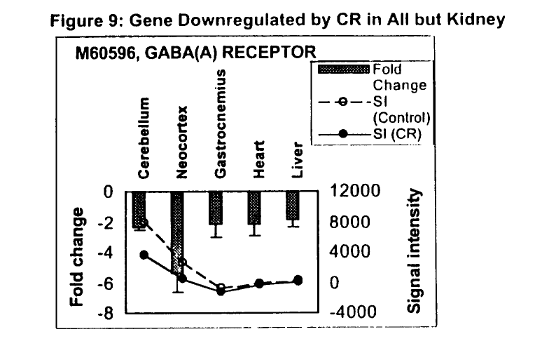

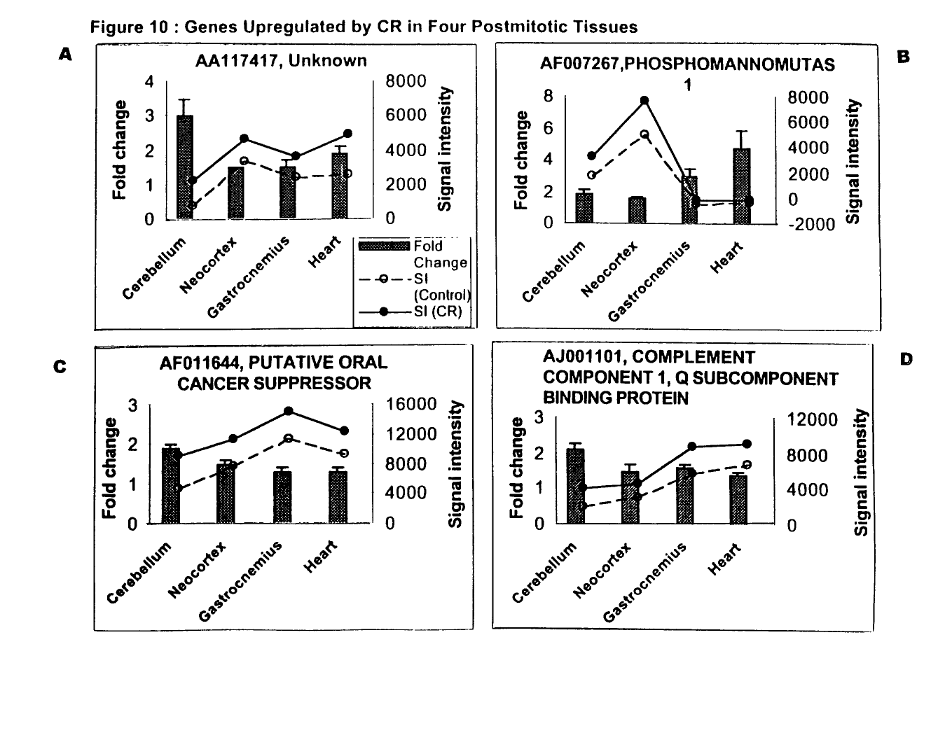

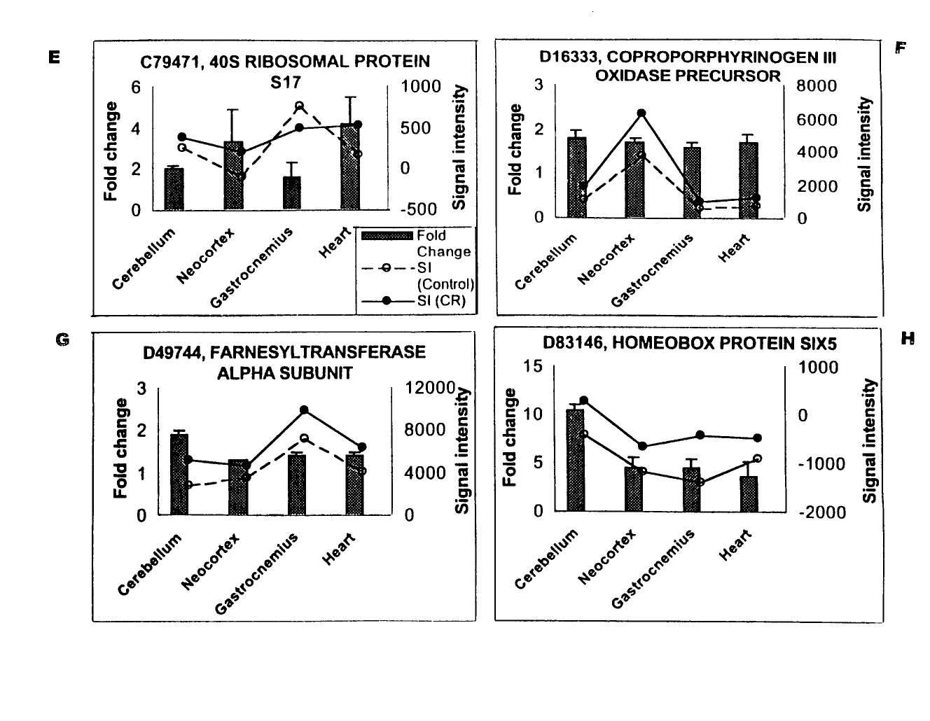

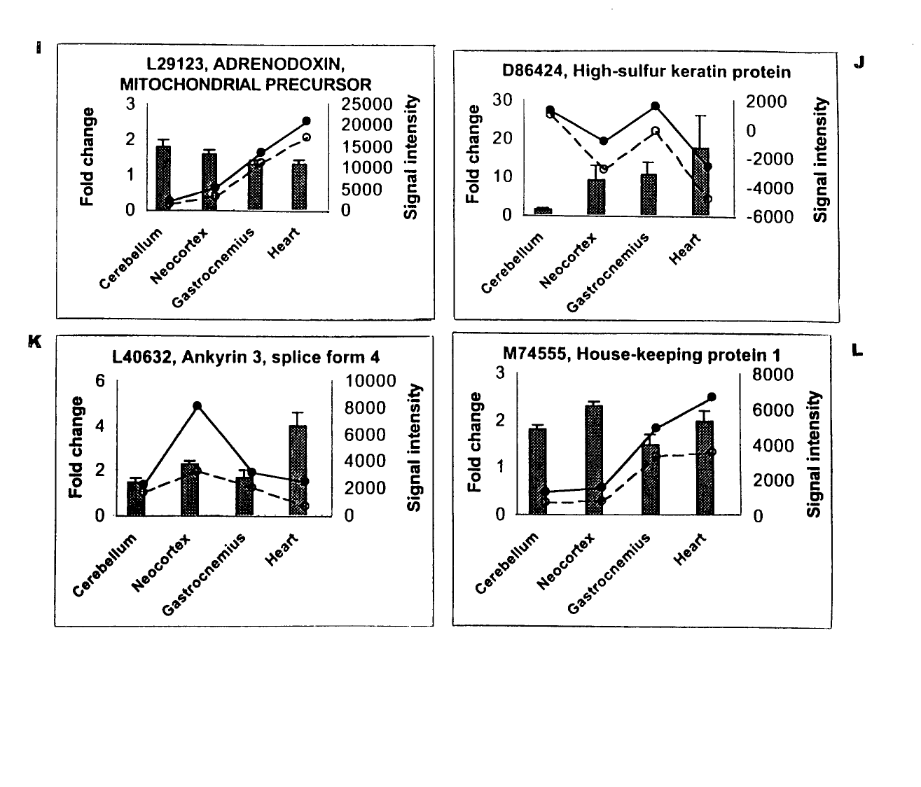

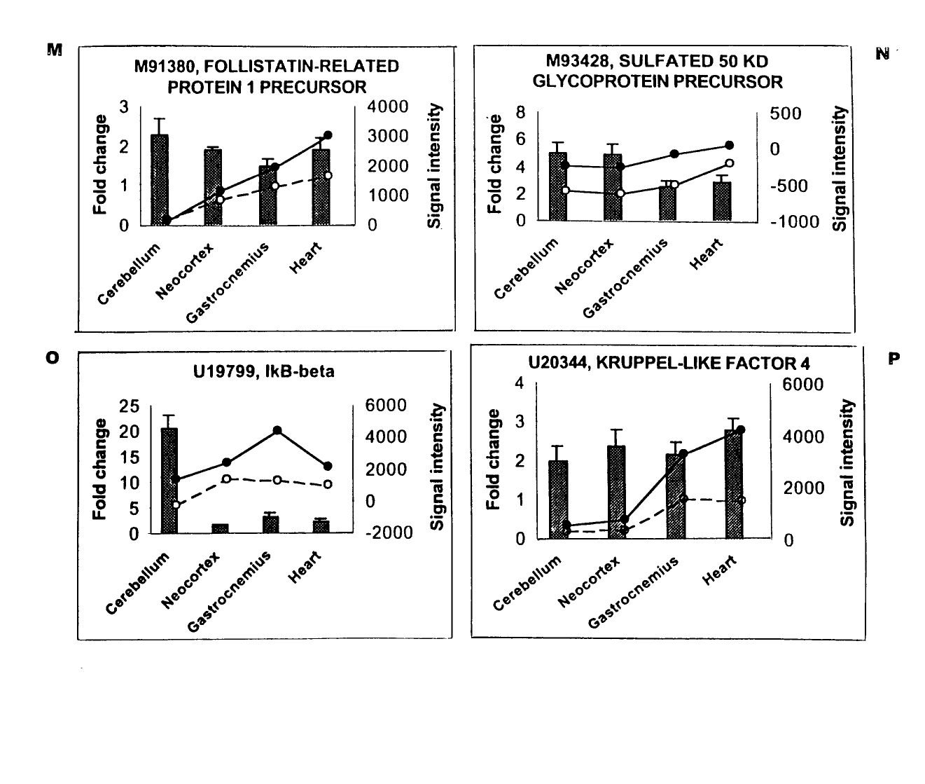

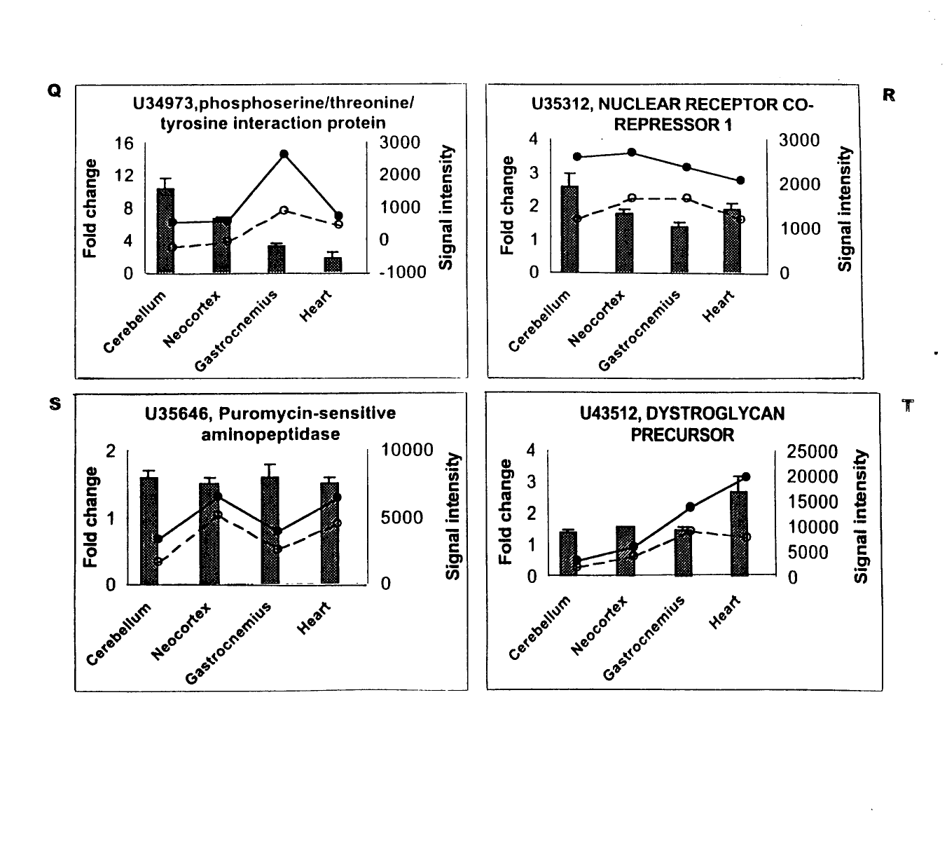

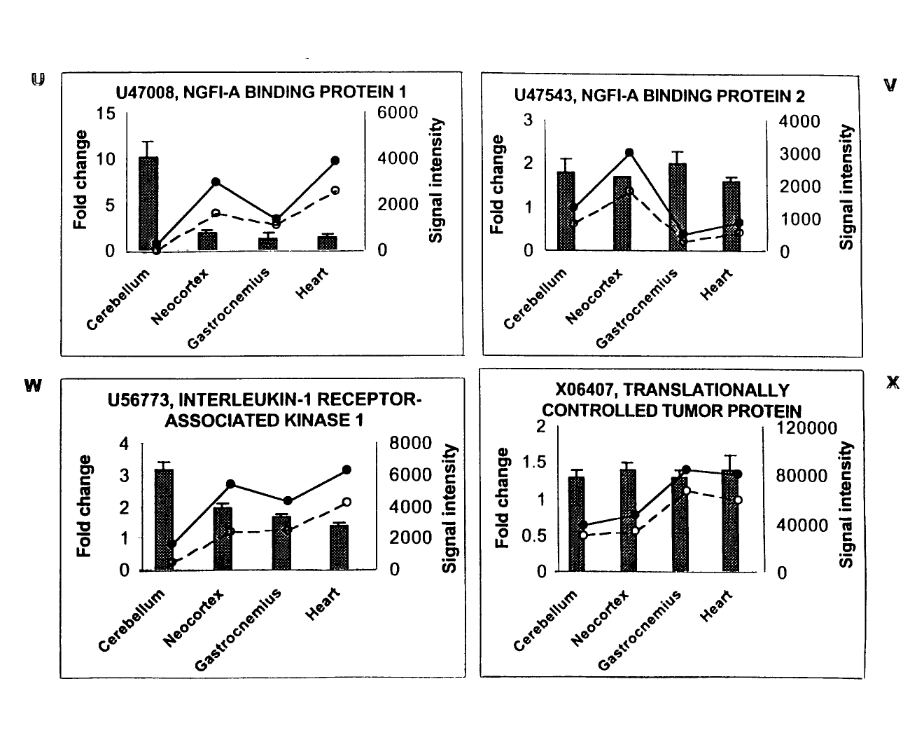

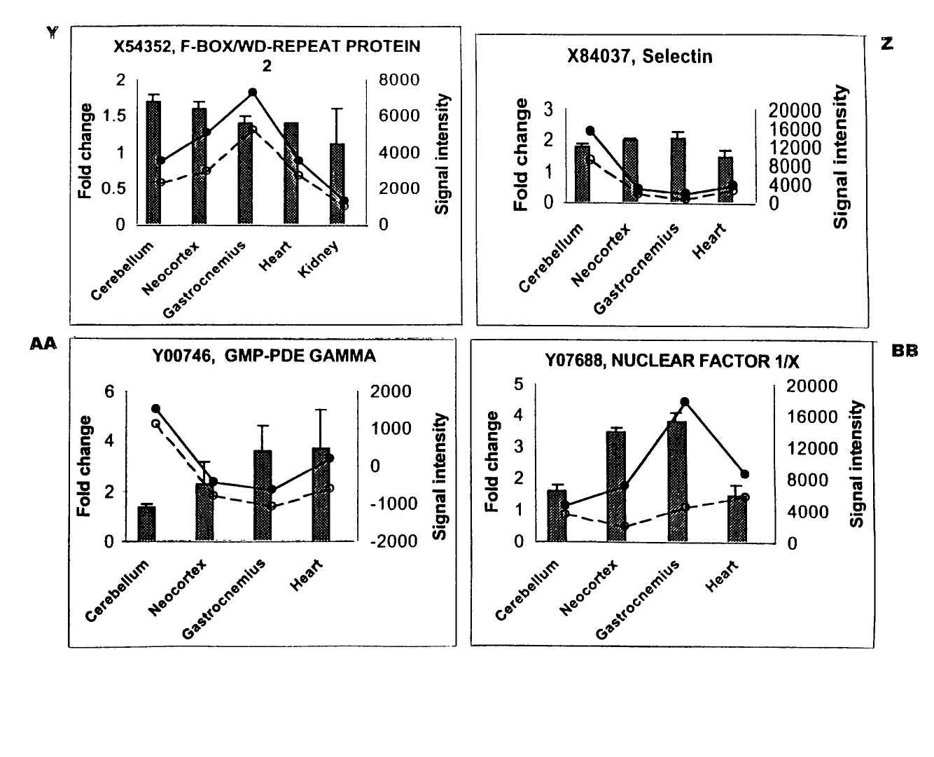

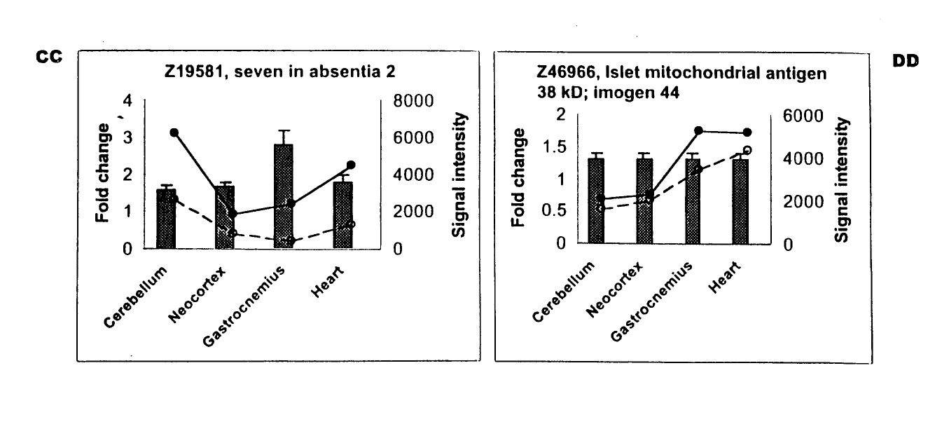

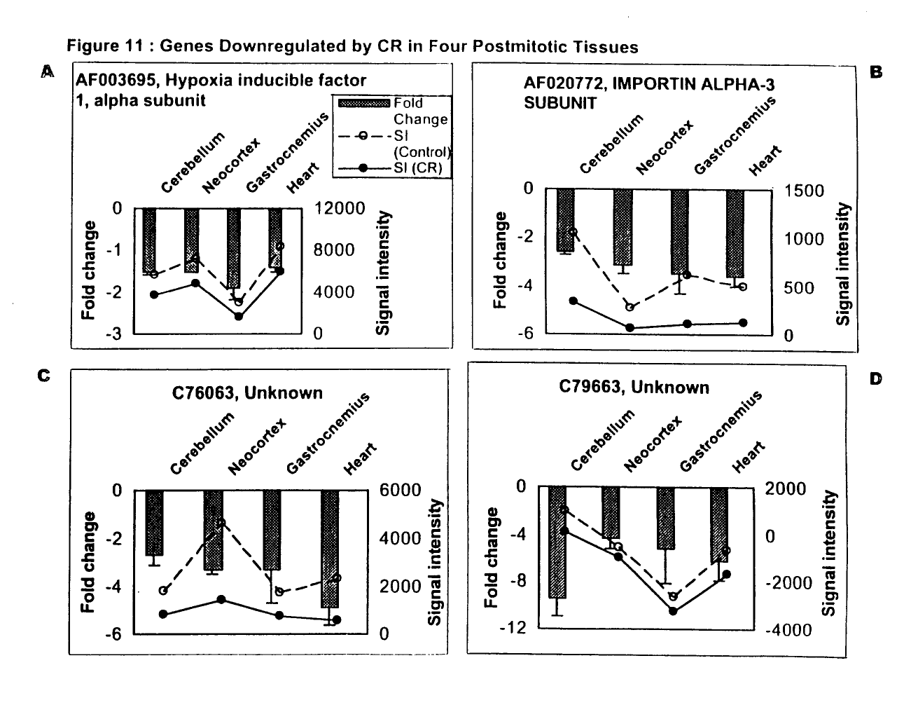

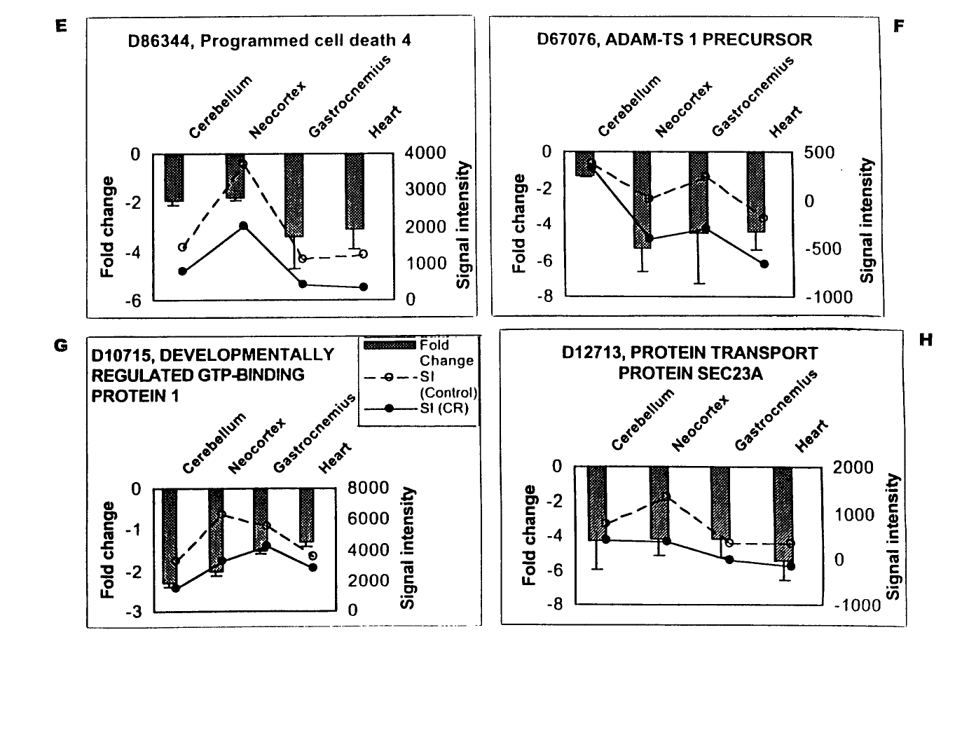

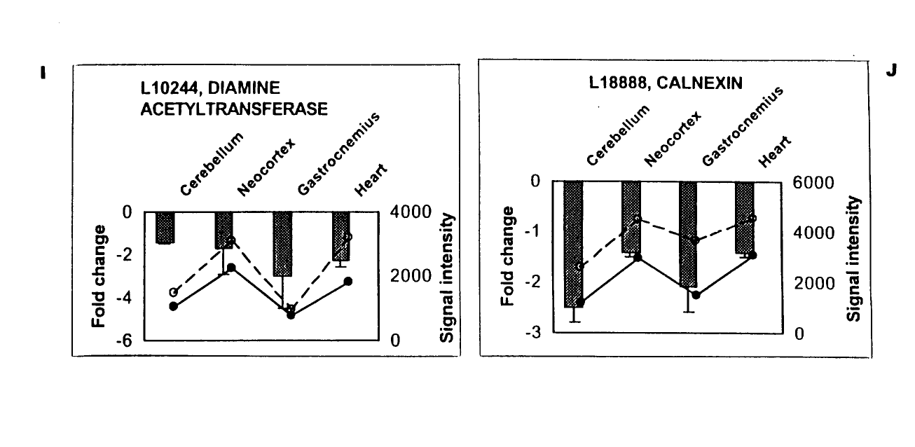

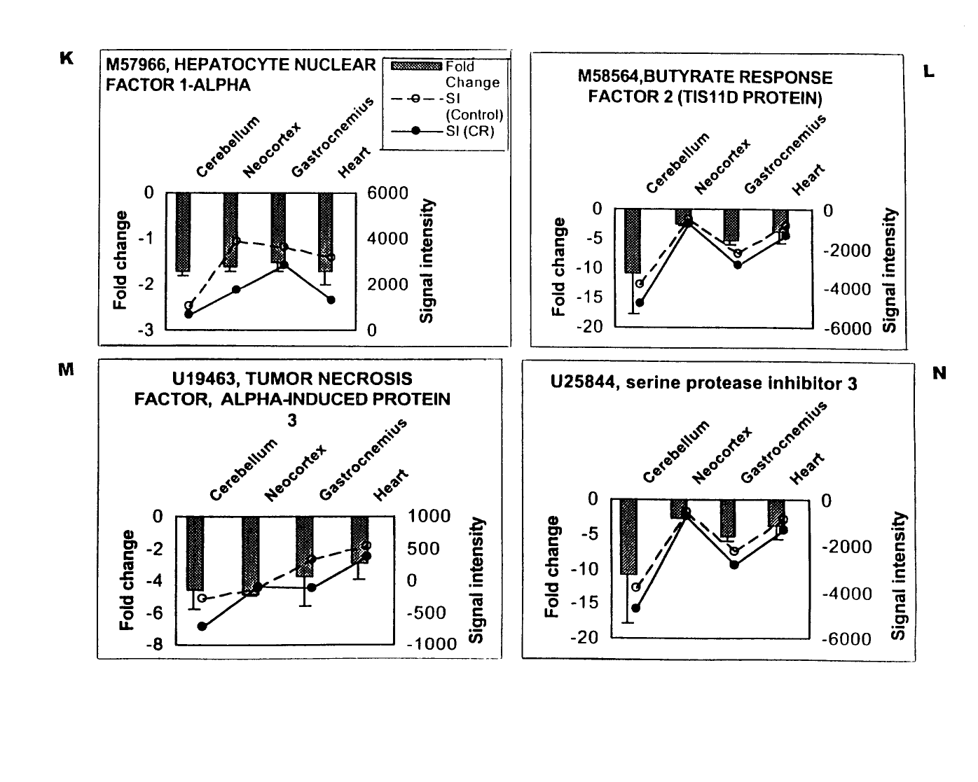

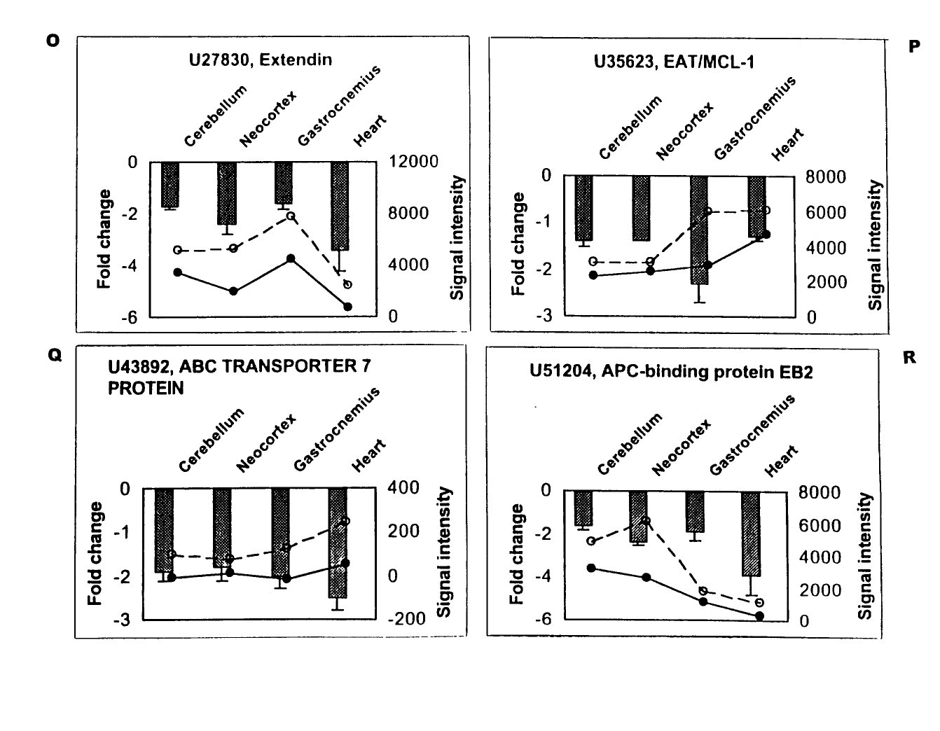

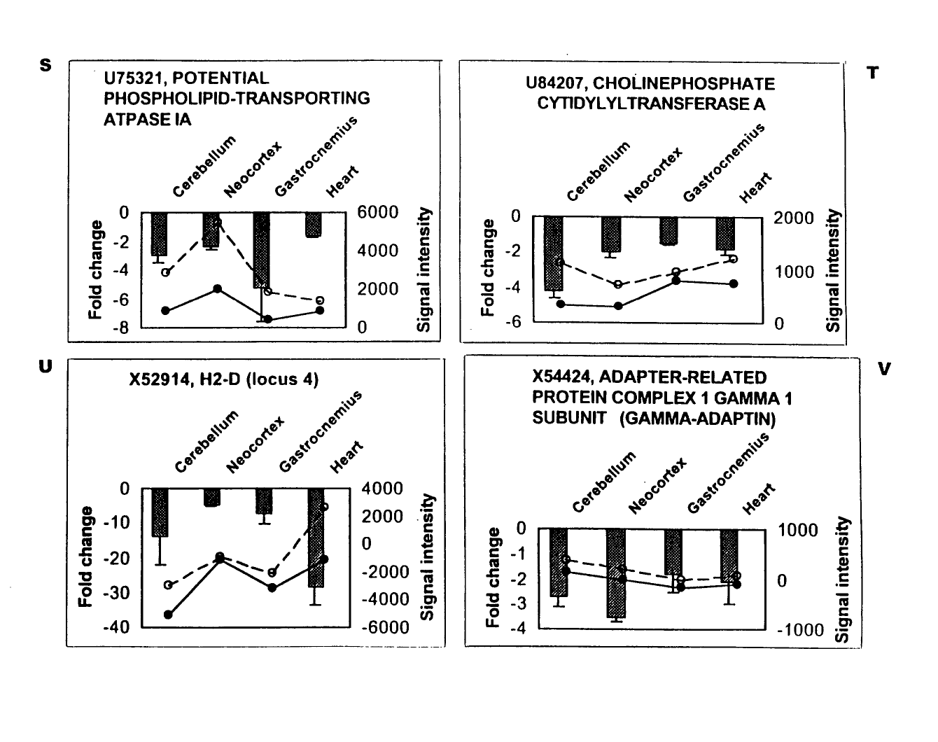

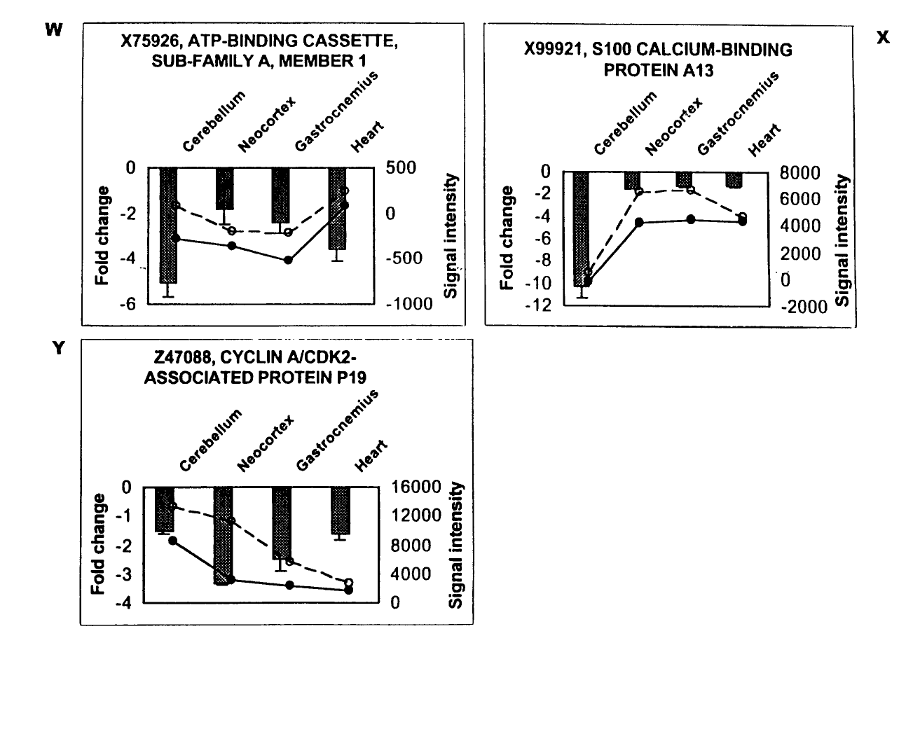

### DESCRIPTION OF THE INVENTION

There exists a large and growing segment of the population in developed countries that is afflicted with age-associated disorders, such as sarcopenia (loss of muscle mass), neurodegenerative conditions, and cardiac diseases. Therefore, the market for compounds that prevent aging-associated disorders and improve the quality of life for the elderly is likely to become a driving force in the research and development of novel drugs by the pharmaceutical industry. Since caloric restriction (CR) is the only established method for retarding aging and age-related diseases in mammals, discovering the genetic and metabolic pathways that are influenced by CR is likely to generate molecular targets for the design of rational interventions. By “caloric restriction” we mean a reduction of caloric intake (typically of 30-50%, depending on animal model) which is obtained without the occurrence of nutrient deficiency (i.e., a state of caloric under-nutrition without malnutrition).

In order to discover interventions that mimic the effects of CR, and therefore retard aging and associated diseases, identification of molecular targets is required. To achieve this goal, we used the U74 and 11 K Affymetrix (Santa Clara, Calif.) murine genome DNA chips to measure the gene expression profile associated with CR for 11,000 genes in six tissues from mice: cerebral cortex, cerebellum, skeletal muscle (gastrocnemius), heart, liver and kidney. Six animals were used per experiment (3 controls and 3 calorie-restricted), resulting in a total of 396,000 independent gene expression measurements including all tissues.

To our knowledge, this study represents the largest search ever performed for gene expression alterations as a function of CR. We reasoned that alterations in gene expression that are shared among 5 to 6 tissues examined, or among the four post-mitotic tissues studied (i.e., cerebellum, neocortex, gastrocnemius and heart), must represent core or fundamental changes associated with CR, as opposed to tissue-specific effects.

In one embodiment, the present invention provides molecular biomarkers of CR. A requirement for the evaluation of genetic, pharmaceutical or nutritional interventions that mimic the effects of CR is the development of CR-related biomarkers. Desirable features for biomarkers of CR are that they should be amenable to quantification and reflect CR-related alterations at the molecular level in the tissue under study. Therefore, the changes in gene expression associated with CR represent targets for pharmaceutical development, gene therapy or RNA antisense therapy with the goal of preventing, retarding or reversing the aging process at the molecular level. These gene expression alterations may also play a role in opposing the development of age-related diseases of the organs under study.

In another embodiment, the invention is a method for measuring the relative metabolic state of a multicellular organism, such as a mammal, at the organ, tissue or cellular level through the characterization of the organism's gene expression patterns. By “relative metabolic state” we mean the comparison of an organism's metabolic state (as measured by the gene expression profile of at least one Table 2 ORF and referred to as the “test profile”) to a CR-treated organism's gene profile and a non-CR treated organism's profile and the determination of which profile is more similar to the test profile. This method preferably comprises obtaining a cDNA copy of the organism's RNA and determining the expression pattern of at least one of the genes listed in Table 2 (genes which change in expression with CR in multiple tissues), preferably at least 5 biomarker sequences, most preferably at least 10 biomarker sequences and more preferably at least 20, 30, 40, or 50 biomarker sequences, within the cDNA. By “gene expression pattern” we mean to include the change in pattern of the encoded RNA or protein.

One may characterize the metabolic state of the organism by determining how many and at what level these genes disclosed are altered in expression. Because the sequences listed in Table 2 are CR-related alterations in multiple tissues, one could use the same sequences to determine the similarity of the gene expression profile induced by an intervention relative to a CR expression profile in multiple tissues, such as, but not limited to, neocortex, heart, cerebellum, kidney, liver and skeletal muscle.

In some embodiments, gene expression is measured by identifying the presence or amount of one or more proteins encoded by one of the genes listed in Table 2.

The present invention also provides systems for detecting two or more markers of interest (e.g., two or more markers from Table 2). For example, where it is determined that a finite set of particular markers provides relevant information, a detection system is provided that detects the finite set of markers. For example, as opposed to detecting all genes expressed in a tissue with a generic microarray, a defined microarray or other detection technology is employed to detect the plurality (e.g., 2, 5, 10, 25) of markers that define a biological condition (e.g., a biological age, a response to a pharmaceutical or diet that increases or decreases rate of aging, etc.).

The present invention is not limited by the method in which biomarkers are detected or measured. In some embodiments, mRNA, cDNA, or protein is detected in tissue samples (e.g., biopsy samples). In other embodiments, mRNA, cDNA, or protein is detected in bodily fluids (e.g., serum, plasma, urine, or saliva). The present invention further provides kits for the detection of biomarkers.

In some preferred embodiments, protein is detected. Protein expression may be detected by any suitable method. In some embodiments, proteins are detected by binding of an antibody specific for the protein. For example, in some embodiments, antibody binding is detected using a suitable technique, including but not limited to, radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, immunoelectrophoresis assays, and proteomic assays, such as the use of gel electrophoresis coupled to mass spectroscopy to identify multiple proteins in a sample.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include, but are not limited to, those described in U.S. Pat. Nos. 5,885,530; 4,981,785; 6,159,750; and 5,358,691, each of which is herein incorporated by reference. In some embodiments, the analysis and presentation of results is also automated. For example, in some embodiments, software that generates a diagnosis and/or prognosis based on the presence or absence of a series of proteins corresponding to markers is utilized.

In other embodiments, the immunoassay described in U.S. Pat. Nos. 5,599,677 and 5,672,480, each of which is herein incorporated by reference, is utilized. In other embodiments, proteins are detected by immunohistochemistry.

In other embodiments, markers are detected at the level of cDNA or RNA. In some embodiments of the present invention, markers are detected using a direct sequencing technique. In these assays, nucleic acid samples are first isolated from a subject using any suitable method. In some embodiments, the region of interest is cloned into a suitable vector and amplified by growth in a host cell (e.g., bacteria). In other embodiments, DNA in the region of interest is amplified using PCR. Following amplification, DNA in the region of interest is sequenced using any suitable method, including but not limited to manual sequencing using radioactive marker nucleotides, or automated sequencing. The results of the sequencing are displayed using any suitable method.

In some embodiments of the present invention, markers are detected using a PCR-based assay. In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In preferred embodiments of the present invention, markers are detected using a hybridization assay. In a hybridization assay, the presence or absence of a marker is determined based on the ability of the nucleic acid from the sample to hybridize to a complementary nucleic acid molecule (e.g., an oligonucleotide probe). A variety of hybridization assays using a variety of technologies for hybridization and detection are available.

In some embodiments, hybridization of a probe to the sequence of interest is detected directly by visualizing a bound probe (e.g., a Northern or Southern assay; See e.g., Ausabel, et al. (eds.), Current Protocols in Molecular Biology, John Wiley & Sons, NY [1991]). In these assays, DNA (Southern) or RNA (Northern) is isolated. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes is allowed to contact the membrane under low, medium, or high stringency conditions. Unbound probe is removed and the presence of binding is detected by visualizing the labeled probe.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given marker are electronically placed at, or “addressed” to, specific sites on the microchip. Since nucleic acid molecules have a strong negative charge, they can be electronically moved to an area of positive charge.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents.

In yet other embodiments, a “bead array” is used for the detection of markers (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given marker. Batches of beads are combined to form a pool specific to the array. To perform an assay, the BEAD ARRAY is contacted with a prepared sample. Hybridization is detected using any suitable method.

In some embodiments of the present invention, hybridization is detected by enzymatic cleavage of specific structures (e.g., INVADER assay, Third Wave Technologies; See e.g., U.S. Pat. Nos. 5,846,717, 6,090,543; 6,001,567; 5,985,557; and 5,994,069; each of which is herein incorporated by reference). In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given marker, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

Additional detection assays that are produced and utilized using the systems and methods of the present invention include, but are not limited to, enzyme mismatch cleavage methods (e.g., Variagenics, U.S. Pat. Nos. 6,110,684; 5,958,692; 5,851,770, herein incorporated by reference in their entireties); branched hybridization methods (e.g., Chiron, U.S. Pat. Nos. 5,849,481; 5,710,264; 5,124,246; and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229; 6,221,583; 6,013,170; and 6,063,573, herein incorporated by reference in their entireties); cycling probe technology (e.g., U.S. Pat. Nos. 5,403,711; 5,011,769; and 5,660,988, herein incorporated by reference in their entireties); ligase chain reaction (Barnay, Proc. Natl. Acad. Sci. USA 88:189-93,1991); and sandwich hybridization methods (e.g., U.S. Pat. No. 5,288,609, herein incorporated by reference in its entirety).

In some embodiments, mass spectroscopy is used to detect markers. For example, in some embodiments, a MassARRAY system (Sequenom, San Diego, Calif.) is used to detect markers (See e.g., U.S. Pat. Nos. 6,043,031; 5,777,324; and 5,605,798; each of which is herein incorporated by reference).

In some embodiments, the present invention provides kits for the identification, characterization, and quantitation of markers. In some embodiments, the kits contain antibodies specific for markers, in addition to detection reagents and buffers. In other embodiments, the kits contain reagents specific for the detection of nucleic acid (e.g., oligonucleotide probes or primers). In preferred embodiments, the kits contain all of the components necessary to perform a detection assay, including all controls, directions for performing assays, and any necessary software for analysis and presentation of results. In some embodiments, the kits contain instructions including a statement of intended use as required by the Environmental Protection Agency or U.S. Food and Drug Administration for the labeling of in vitro diagnostic assays and/or of pharmaceutical or food products.

Comparison of the organism's gene expression pattern with the result expressed in Table 2 would indicate whether the organism has an aberrant gene expression profile which may indicate that the organism is metabolically similar to a CR-treated animal.

In another embodiment, the present invention is a method of screening a test compound for the ability to inhibit, retard, reverse or mimic the CR process in mammalian tissue. In a typical example of this embodiment, one would first treat a test mammal with a test compound and then analyze a representative tissue of the mammal for the level of expression of the genes or sequences which change in expression in response to CR (Table 2). Preferably, the tissue is selected from the group consisting of brain tissue, heart tissue, muscle tissue, skeletal muscle, kidney, heart and liver tissue. One then compares the analysis of the tissue with a control, untreated mammal and identifies test compounds that are capable of modifying the expression of the biomarker sequences in the mammalian samples such that the expression is indicative of CR-treated tissue.

As an example, a group of young rodents (e.g., mice) would be divided into a control group and a test group. The test group would receive a test compound, such as a dietary supplement, added to food from age 7 weeks to 5 months, whereas the control group would receive a standard diet without the compound during this time period. At age 5 months, several tissues would be collected from animals from each group and a gene expression profile of at least one of the genes listed in Table 2 (preferably at least five genes) would be obtained and would be compared to the profile of control animals. One would then determine whether, for any of the organs investigated, the gene expression pattern of the animals receiving the test compound was more similar to that of CR animals or to the animals on a normal diet.

In another embodiment of the present invention, one would use the sequences of the genes disclosed in Table 2 for a therapy for mimicking the CR metabolic state. In general, one would try to amplify gene expression for the genes identified herein as increasing during CR process and decrease the expression of genes identified herein as decreasing during the CR process. For example, one might try to decrease the expression of genes or sequences identified in Table 2 as decreasing in all 6 tissues. One might attempt to increase the expression of the genes identified in Table 2 as increasing in all 6 tissues. Other preferred transcripts or sequences would be U84411, U51167, U43836, U60001, D49473, L08651, U28917, X59846, AA117417, AF011644, AJ001101, D16333, D49744, L29123, M74555, U19799, U20344, U35312, U43512, U47543, U56773, X54352, Z19581, AF003695, C76063, D10715, D12713, D86344, L18888, U27830, U43892, U51204, U75321, X54424, and Z47088. Methods of increasing and decreasing expression would be known to one of skill in the art. Examples for supplementation of expression would include supplying the organism with additional copies of the gene. A preferred example for decreasing expression would include RNA antisense technologies or pharmaceutical intervention.

The genes disclosed in Table 2 would be appropriate drug development targets. One would use the information presented in the present application for drug development by using currently existing, or by developing, pharmaceutical compounds that either mimic or inhibit the activity of the genes listed in Table 2, or the proteins encoded by these genes.

Therefore, the biomarker genes disclosed herein represent targets for pharmaceutical development and gene therapy or RNA antisense therapy with the goal of mimicking the CR process at the molecular level. These gene expression alterations may also play a role in age-related diseases of the organs under study. Additionally, these genes represent biomarkers of the aging process that can be used for diagnostic purposes.

In a particularly preferred form of the present invention, the targeted genes or proteins would be encoded by ORFs D31966, R74626, U79163, M22531, U43285, U79523, X81059, and X84239.

The present invention further provides methods for selecting subjects (e.g., humans and animals) that are appropriate targets for a particular therapy. In some such embodiments, a sample from the subject is tested for one or more markers (e.g., markers in Table 2). The expression profile of the subject is then used to select a therapy appropriate for that individual's specific condition.

The present invention also provides expression profiles. In some such embodiments, a test sample is assayed for the presence of one or more biomarkers and compared to the expression profile, for example, to determine the relative metabolic state of the sample and/or to determine the effect of a diet or other therapy on the sample. The present invention is not limited by the form of the expression profile. In some embodiments, the expression profile is maintained in computer software. In some embodiments, the expression profile is written material. The present invention is not limited by the number of markers provided or displayed in an expression profile. For example, the expression profile may comprise two or more markers found in Table 2, indicating a biological status of a sample.

The present invention further provides databases comprising expression information (e.g., expression profiles comprising one or more markers from Table 2 from one or more samples). In some embodiments, the databases find use in data analysis, including, but not limited to, comparison of markers to one or more public or private information databases (e.g., OMIM, GenBank, BLAST, Molecular Modeling Databases, Medline, genome databases, etc.). In some such embodiments, an automated process is carried out to automatically associate information obtained from data obtained using the methods of the present invention to information in one or more of public or private databases. Associations find use, for example, in making expression correlations to phenotypes (e.g., disease states).

The present invention also provides methods for selecting ingredients in food or dietary products (e.g., nutraceuticals) and food and dietary products thus generated. For example, a food or dietary product is altered (e.g., supplemented or depleted) with a factor that increases or decreases, directly or indirectly, the expression of one or more age-related markers (e.g., markers in Table 2). In some embodiments, the food or dietary product is altered with a factor that might increase or decrease, directly or indirectly, the expression of one or more CR-related markers (e.g., markers in Table 2).

We also understand the present invention to be extended to mammalian homologs of the mouse genes listed in Table 2. One of skill in the art could easily investigate homologs in other mammalian species by identifying particular genes with sufficiently high homology to the genes listed in Table 2. By “high homology” we mean that the homology is at least 50% overall (within the entire gene or protein) either at the nucleotide or amino acid level.

### EXAMPLES

Preferred Methods

A. Animal ages, husbandry and dietary manipulations. All aspects of animal care were approved by the appropriate committees and conformed with institutional guidelines. Details on the methods employed to house and feed male B6 mice, a commonly used model in aging research with an average lifespan of ˜30 months, were described (Pugh, et al., 1999). Briefly, mice were purchased from Charles River Laboratories (Wilmington, Mass.) at 1.5 months of age. After receipt in Madison, the mice were housed singly in the specific pathogen-free Shared Aging Rodent Facility at the Madison Va. Geriatric Research, Education and Clinical Center, and provided a nonpurified diet (PLI 5001 [Purina Labs, St. Louis, Mo.]) and acidified water ad libitum for one week.

At ˜7 weeks of age, each mouse was individually caged and fed in a calorie-controlled manner as described by Pugh, et al. (1999). Two semipurified, nearly isocaloric (˜4.1 kcal/g) powdered diets made by Teklad, Inc. (Madison, Wis.) were used. The diet termed “Restricted” (R), cat. #91351, was designed to be fed at ˜75% of the level of the “Normal” (N) diet, cat. #91349. At this reduced intake level, the R diet supplies 25% fewer calories, mainly through a 13% reduction in the intake of two carbohydrate components, sucrose and cornstarch. The protein (casein), minerals and vitamins are enriched in the R diet such that nearly identical amounts of these components are fed to both N and R animals after a 25% reduction in diet. The fat component, corn oil, is the same for both diets, leading to a 25% reduction in fat intake when feeding the R diet. In this way we place the mouse in a healthful state of undernutrition without malnutrition.

B. Gene expression analysis. At 5 months of age, the mice were euthanized by rapid cervical dislocation and organs harvested, placed in microcentrifuge tubes, immediately flash-frozen in liquid nitrogen and stored at −80° C. All experiments used three mice per experimental group (i.e., control and CR). RNA from each animal was independently hybridized to DNA chips, so that intragroup variability is known. Our own data indicate that variability between animals in the same age/diet group is minimal, since we have never observed correlation coefficients between two animals to be <0.98. Mice were autopsied to exclude animals showing overt disease and, given that young mice were studied, none was detected.

Total RNA was extracted from frozen tissue using TRIZOL reagent (Life Technologies) and a power homogenizer (Fisher Scientific) with the addition of chloroform for the phase separation before isopropyl alcohol precipitation of total RNA. Poly (A)+ RNA is purified from the total RNA with oligo-dT linked Oligotex resin (Qiagen). Two micrograms of poly (A)+ RNA are converted into double-stranded cDNA (ds-cDNA) using SuperScript Choice System (Life Technologies) with an oligo dT primer containing a T7 RNA polymerase promoter region (Genset). After second strand synthesis, the reaction mixture is extracted with phenol/chloroform/isoamyl alcohol. Phase Lock Gel (5 Prime 3 Prime, Inc.) is used to increase ds-cDNA recovery. The ds-cDNA is collected by ethanol precipitation. The pellet is resuspended in 3 μl of DEPC-treated water. In vitro transcription is performed using a T7 Megascript Kit (Ambion) with 1.5 μl of ds-cDNA template in the presence of a mixture of unlabeled ATP, CTP, GTP, and UTP and biotin-labeled CTP and UTP (bio-11-CTP and bio-16-UTP [Enzo]). Biotin-labeled cRNA is purified using a Rneasy affinity column (Qiagen). The amount of biotin-labeled CRNA is determined by measuring absorbency at 260 nm. Biotin-labeled cRNA is fragmented randomly to sizes ranging from 35 to 200 bases by incubating at 94° C. for 35 minutes in 40 mM Trisacetate pH 8.1, 100 mM potassium acetate, and 30 mM magnesium acetate. The hybridization solutions contain 100 mM MES, 1 M [Na+], 20 mM EDTA, and 0.01% Tween 20. The hybridization solutions also contained 50 pM oligonucleotide B2 (a biotin-labeled control oligonucleotide used for making grid alignments), 0.1 mg/mL herring sperm DNA, and 0.5 mg/mL acetylated BSA. The final concentration of fragmented cRNA is 0.05 μg/μl in the hybridization solutions. Hybridization solutions are heated to 99° C. for 5 minutes followed by 45° C. for 5 minutes before being placed in the gene chip. 10 μg of cRNA is placed in the gene chip. Hybridizations were carried out at 45° C. for 16 hours with mixing on a rotisserie at 60 rpm. Following hybridization, the hybridization solutions are removed and the gene chips installed in a fluidics system for wash and stain. The fluidics system (Affymetrix GeneChip Fluidics Station 400) performs two post hybridization washes (a non-stringent wash and a stringent wash), staining with streptavidin-phycoerythrin, and one post-stain wash. The gene chips are read at a resolution of 6 μm using a Hewlett Packard GeneArray Scanner. Data collected from two scanned images are used for the analysis.

C. Data analysis performed by Affymetrix® software. Detailed protocols for data analysis of Affymetrix microarrays and extensive documentation of the sensitivity and quantitative aspects of the method have been described (Lockhart, et al., 1996). The U74 series is derived from UniGene (http://www.ncbi.nlm.nih.gov/UniGene/). Briefly, each gene is represented by the use of ˜20 perfectly matched (PM) and an equal number of mismatched (MM) control probes. The MM probes act as specificity controls that allow the direct subtraction of both background and cross-hybridization signals. The number of instances in which the PM hybridization signal is larger than the MM signal is computed along with the average of the logarithm of the PM:MM ratio (after background subtraction) for each probe set. These values are used to make an arbitrary matrix-based decision concerning the presence or absence of an RNA molecule, which serves as an indicator of data quality. All calculations are performed by Affymetrix software. To determine the quantitative RNA abundance, the average of the differences representing PM minus MM for each gene-specific probe family is calculated, after discarding the maximum, the minimum, and any outliers beyond three standard deviations. This value, termed the Average Intensity Difference (SI), is a function of mRNA abundance. In order to make comparisons between data sets, the Average Intensity Differences for each gene are normalized to the total fluorescence intensity of the array. This is similar to the concept of normalizing signal to a reference mRNA, such as p-actin in a typical Northern blot.

In order to calculate fold changes (FC) between data sets (after normalization) obtained from restricted (r) vs. control (c) vs. mice, the following formula is used by the software:

FC=SIᵣ−SI_{c}+1 if SIᵣ≧SI_{c} or −1 if SIᵣ<SI_{c}

the smallest of either SIᵣ or SI_{c}

Where SIᵣ is the average signal intensity from a gene-specific probe family from a calorie-restricted mouse and SI_{c} is that from a control mouse. Alternatively, if the Q_{factor}, a measure of the non-specific fluorescence intensity background, is larger than the smallest of either SI_{c} or SIᵣ, the FC is calculated as:

FC=SIᵣ−SI_{c}

Q_{factor}

The Q_{factor} is automatically calculated for different regions of the microarray and, therefore, minimizes the calculation of spurious fold changes. Average of pairwise comparisons are made between study groups, each composed of three animals, using Excel software. For example, each tissue from a 5-month-old control mouse (n=3) is compared to a 5-month-old calorie-restricted mouse (n=3), generating a total of 9 pairwise comparisons for each of the six tissues being studied.

D. Numbers of genes selected for inclusion in this patent application. The numbers of genes identified showing shared changes in expression with CR in 5-6 of the tissues examined are summarized in Table 1. We have also included the genes that showed either upregulation or downregulation in all four tissues studied that are composed mainly of postmitotic (non-dividing) cells: gastrocnemius, heart, cerebellum and neocortex. The procedure involved a computer search of our database to identify those genes which showed 1.1-fold or greater increases or decreases in expression with CR in either five or all six of the tissues examined. The data supporting the change were then critically evaluated for data quality based on information provided by Affymetrix software as well as signal intensity (which also provides information on tissue-specific expression levels), and variation (standard error). In order to be accepted for inclusion, genes had to show an increase or decrease in expression that was >1.1-fold+1 SEM as determined for the 9 pairwise comparisons between the three animals in each experimental group. The genes within each group are listed in descending alphabetical order of the GenBank accession codes.

Shared Changes in Gene Expression with Caloric Restriction

A. Synopsis. Table 1 provides an overview of the changes in gene expression associated with CR which were shared among the six tissues studied. Of the 162 genes that showed an increase or decrease in expression only 84 (52%) were accepted for further analysis.

TABLE 1
Overview of the Genes Meeting Criteria for Selection

| | Upregulated with CR | | Downregulated with CR | |
|---|---|---|---|---|
| Number of Tissues | Accept | Reject | Accept | Reject |
| 6 | 3 | 2 | 5 | 7 |
| 5 minus Cerebellum | 0 | 1 | 0 | 4 |
| 5 minus Gastroc. | 4 | 5 | 1 | 7 |
| 5 minus Heart | 3 | 1 | 2 | 8 |
| 5 minus Kidney | 5 | 3 | 1 | 8 |
| 5 minus Liver | 5 | 2 | 0 | 7 |
| 5 minus Neocortex | 0 | 2 | 0 | 4 |
| 4 Post-mitotics | 30 | 4 | 25 | 14 |
| Totals | 50 | 20 | 34 | 58 |

Summary
Total Genes Initially Selected 162
Total Genes Finally Accepted (%) 84 (52%)
% of Accepted Genes Going Up with CR 59%
% of Accepted Genes Going down with CR 41%
% Selected among genes going up with CR (all tissues) 71%
% Selected among genes going down with CR (all tissues) 37%
% Selected among genes going up with CR (post-mitotics) 88%
% Selected among genes going down with CR (post-mitotics) 64%

TABLE 2
Genes Displaying Shared Changes Induced by CR in Multiple Tissues

| | | | | | | | |
|---|---|---|---|---|---|---|---|
| ORF | Gene | Crebell. | Neocrtx. | Gastroc. | Heart | Kidney | Liver |

Up with CR in All Six Tissues

| | | | | | | | |
|---|---|---|---|---|---|---|---|
| D31966 | RNA polymerase I 40KD subunit | 1.4 (01.) | 5.8 (0.8) | 3.4 (1.4) | 6.1 (2.1) | 1.3 (0.1) | 1.5 (0.2) |
| R74626 | Unknown (no homology >33%) | 30.1 (2.4) | 1.5 (0.1) | 1.2 (0.1) | 1.3 (0.1) | 3.4 (2.3) | 12.1 (2.1) |
| U79163 | Noggin precursor | 5.0 (1.8) | 1.2 (0.1) | 1.3 (0.1) | 1.5 (0.1) | 5.5 (1.1) | 1.9 (0.7) |

Down with CR in All Six Tissues

| | | | | | | | |
|---|---|---|---|---|---|---|---|
| M22531 | Complement C1qB | −1.8 (0.2) | −3.7 (0.6) | −2.5R (0.9) | −6.0 (1.4) | −1.4 (0.1) | −1.9 (0.5) |
| U43285 | Selenide, water dikinase 2 (Selenophosphate synthetase | −2.1 (0.1) | −5.8 (0.6) | −2.7 (0.5) | −3.4 (0.7) | −1.3 (0.0) | −1.7 (0.2) |
| | 2) | | | | | | |
| U79523 | Peptidylglycine alpha-amidating monooxygenase | −2.2 (0.5) | −5.9 (2.0) | −4.5 (2.1) | −1.9 (0.4) | −3.3 (0.5) | −2.0 (0.8) |
| X81059 | teg271 (testes-expressed gene 271) | −1.7 (0.2) | −1.9 (0.0) | −6.2 (2.4) | −7.9 (3.2) | −1.5 (0.1) | −3.5 (0.8) |
| X84239 | Rab5b | −3.9 (2.0) | −3.5 (0.6) | −4.5 (2.2) | −12.9 (1.4) | −2.3 (0.7) | −2.2 (0.9) |
| | Protein transport | | | | | | |

Up with CR in Five of Six Tissues
Up with CR in all but Cerebellum: none met criteria
Up with CR in all but Gastrocnemius

| | | | | | | |
|---|---|---|---|---|---|---|
| D38117 | m-calpain (large subunit) | 2.4 (0.1) | 1.6 (0.0) | 1.8 (0.2) | 2.6 (0.3) | 2.9 (1.2) |
| M70642 | Connective tissue growth factor precursor (CTGF) (FISP- | 3.4 (0.2) | 2.0 (0.2) | 2.3 (0.3) | 1.4 (0.1) | 2.7 (0.5) |
| | 12 protein) | | | | | |
| U37775 | Tuberin (tuberous sclerosis 2 homolog protein) | 1.3 (0.1) | 1.4 (0.0) | 1.5 (0.2) | 2.2 (0.5) | 1.8 (0.7) |
| U84411 | Protein tyrosine phosphatase type IVA, member 1; | 1.3 (0.1) | 1.3 (0.0) | 1.5 (0.0) | 1.7 (0.2) | 1.4 (0.1) |
| | Protein tyrosine phosphatase IVA1 | | | | | |

Up with CR in all but Heart

| | | | | | | |
|---|---|---|---|---|---|---|
| D87117 | Presynaptic protein SAP102 (synapse-associated protein | 1.3 (0.1) | 4.7 (2.0) | 5.0 (1.5) | 2.8 (0.3) | 1.9 (0.7) |
| | 102) (discs, large homolog 3) | | | | | |
| U31966 | Carbonyl reductase | 1.3 (0.1) | 1.5 (0.2) | 2.8 (1.0) | 1.6 (0.1) | 1.5 (0.1) |
| U51167 | Isocitrate dehydrogenase 2 | 1.5 (0.0) | 1.6 (0.1) | 1.5 (0.2) | 1.3 (0.1) | 2.4 (0.3) |

Up with CR in all but Kidney

| | | | | | | |
|---|---|---|---|---|---|---|
| M97900 | Pink-eyed dilution | 2.4 (1.1) | 3.7 (1.7) | 6.8 (1.8) | 2.4 (1.2) | 3.6 (1.1) |
| U32684 | Serum paraoxonase/arylesterase 1 (PON 1) (serum | 3.6 (0.5) | 2.8 (0.5) | 1.9 (0.3) | 2.9 (1.2) | 1.2 (0.1) |
| | aryldialkylphosphatase 1) (A-esterase 1) (aromatic | | | | | |
| | esterase 1) | | | | | |
| U43836 | Vascular endothelial growth factor B precursor | 2.2 (0.3) | 1.8 (0.1) | 1.7 (0.3) | 1.8 (0.3) | 2.0 (0.6) |
| | (VEGF-B) | | | | | |
| U60001 | Histidine triad nucleotide-binding protein (protein kinase | 1.5 (0.1) | 1.6 (0.1) | 1.6 (0.1) | 1.4 (0.1) | 1.8 (0.2) |
| | C inhibitor 1) (protein kinase c-interacting protein 1) | | | | | |
| | (PKCI-1) | | | | | |
| X61450 | Brain protein E161 | 1.9 (0.2) | 1.9 (0.1) | 1.8 (0.3) | 2.5 (1.2) | 2.1 (1.0) |

Up with CR in all but Liver

| | | | | | | |
|---|---|---|---|---|---|---|
| D49473 | Transcription factor SOX-17 | 1.9 (0.3) | 2.3 (0.2) | 1.3 (0.1) | 1.5 (0.1) | 1.2 (0.5) |
| L08651 | 60S Ribosomal protein L29 | 2.0 (0.1) | 1.3 (0.0) | 1.7 (0.3) | 1.6 (0.2) | 1.3 (0.1) |
| U28917 | 60S Ribosomal protein L13 (A52) | 1.3 (0.1) | 1.6 (0.1) | 1.6 (0.2) | 1.4 (0.0) | 1.4 (0.1) |
| U49507 | Lisch7 | 13.1 (1.6) | 2.5 (1.3) | 4.0 (1.0) | 4.5 (1.1) | 3.4 (1.3) |
| X59846 | Growth arrest specific 6 | 1.4 (0.1) | 1.6 (0.0) | 1.4 (0.1) | 1.3 (0.1) | 1.4 (0.1) |

Up with CR in all but Neocortex: None met criteria
Down with CR in Five of Six Tissues
Down with CR in all but Cerebellum: None met criteria
Down with CR in all but Gastrocnemius

| | | | | | | |
|---|---|---|---|---|---|---|
| X00958 | H2-class II, E-B beta chain precursor | −1.7 (0.1) | −2.2 (0.9) | −2.8 (1.0) | −2.0 (1.0) | −6.1 (3.2) |

Down with CR in all but Heart

| | | | | | | |
|---|---|---|---|---|---|---|
| K03235 | Proliferin 2 precursor (mitogen-regulated protein 2) | −6.4 (1.6) | −3.7 (2.2) | −4.8 (0.7) | −3.6 (0.9) | −2.9 (0.6) |
| Z48238 | Hypothetical protein (B2 element) - 73% homol. To | −1.9 (0.1) | −2.2 (0.3) | −1.3 (0.1) | −1.3 (0.1) | −2.3 (0.8) |
| | small part | | | | | |

Down with CR in all but Kidney

| | | | | | | |
|---|---|---|---|---|---|---|
| M60596 | Gamma-aminobutyric-acid receptor delta subunit | −2.3 (0.2) | −5.4 (1.2) | −2.1 (0.9) | −2.1 (0.8) | −1.9 (0.4) |
| | precursor (GABA(A) receptor) | | | | | |

Down with CR in all but Liver: None met criteria
Down in all but Neocortex: None met criteria
Up with CR in Four Postmitotic Tissues

| | | | | | |
|---|---|---|---|---|---|
| AA117417 | Unknown (no homology >37%) | 3.0 (0.5) | 1.5 (0.0) | 1.5 (0.2) | 1.9 (0.2) |
| AF007267 | Phosphomannomutase 1 (PMM 1) | 1.9 (0.2) | 1.6 (0.1) | 3.0 (0.4) | 4.7 (1.2) |
| AF011644 | Putative oral cancer suppressor (deleted in oral cancer-1) | 1.9 (0.1) | 1.5 (0.1) | 1.3 (0.1) | 1.3 (0.1) |
| | (DOC-1) | | | | |
| AJ001101 | Complement component 1, Q subcomponent binding | 2.1 (0.2) | 1.5 (0.2) | 1.6 (0.1) | 1.4 (0.1) |
| | protein, mitochondrial precursor (glycoprotein GC1QBP) | | | | |
| | (GC1Q-R protein) | | | | |
| C79471 | 40S ribosomal protein S17 (83% homol.) | 2.0 (0.2) | 3.3 (1.6) | 1.6 (0.7) | 4.2 (1.3) |
| D16333 | Coproporphyrinogen III OXIDASE precursor | 1.8 (0.2) | 1.7 (0.1) | 1.6 (0.1) | 1.7 (0.2) |
| | (coproporphyrinogenase) (coprogen oxidase) | | | | |
| D49744 | Farnesyltransferase alpha subunit (CAAX | 1.9 (0.1) | 1.3 (0.0) | 1.4 (0.1) | 1.4 (0.1) |
| | farnesyltransferase alpha subunit) (FTASE-alpha) | | | | |
| D83146 | Homeobox protein six5 | 10.4 (0.6) | 4.5 (1.1) | 4.6 (0.9) | 3.6 (1.5) |
| D86424 | High-sulfur keratin protein | 1.5 (0.3) | 9.2 (4.0) | 10.7 (3.2) | 17.8 (8.3) |
| L29123 | Adrenodoxin, mitochondrial precursor (adrenal | 1.8 (0.2) | 1.6 (0.1) | 1.3 (0.1) | 1.3 (0.1) |
| | ferredoxin) | | | | |
| L40632 | Ankyrin 3, splice form 4 | 1.5 (0.2) | 2.3 (0.1) | 1.7 (0.3) | 4.0 (0.6) |
| M74555 | House-keeping protein 1 | 1.8 (0.1) | 2.3 (0.1) | 1.5 (0.2) | 2.0 (0.2) |
| M91380 | Follistatin-related protein 1 precursor (TGF-beta- | 2.3 (0.4) | 1.9 (0.1) | 1.5 (0.2) | 1.9 (0.3) |
| | inducible protein TSC-36) | | | | |
| M93428 | Sulfated 50 KD glycoprotein precursor (SGP50) | 5.0 (0.8) | 4.9 (0.8) | 2.5 (0.5) | 2.9 (0.5) |
| | (endothelial ligand for I-selectin) (glycosylation- | | | | |
| | dependent cell adhesion molecule 1) (GLYCAM-1) | | | | |
| | (MC26) | | | | |
| U19799 | IkB-beta | 20.7 (2.6) | 1.8 (0.1) | 3.2 (0.9) | 2.3 (0.5) |
| U20344 | Kruppel-like factor 4 (gut enriched Kruppel-like factor) | 2.0 (0.4) | 2.4 (0.4) | 2.2 (0.3) | 2.8 (0.3) |
| | (epithelial zinc-finger protein EZF) | | | | |
| U34973 | Phosphoserine/threonine/tyrosine interaction protein; | 10.4 (1.3) | 6.7 (0.3) | 3.2 (0.5) | 1.8 (0.7) |
| | protein tyrosine phosphatase-like unspliced c-terminal | | | | |
| | product and spliced c-terminal end STYX | | | | |
| U35312 | Nuclear receptor co-repressor 1 (N-COR1) (N-COR) | 2.6 (0.4) | 1.8 (0.1) | 1.4 (0.1) | 1.9 (0.2) |
| | (retinoid X receptor interacting protein 13) (RIP13) | | | | |
| U35646 | Puromycin-sensitive aminopeptidase | 1.6 (0.1) | 1.5 (0.1) | 1.6 (0.2) | 1.5 (0.1) |
| U43512 | Dystroglycan precursor (dystrophin-associated | 1.4 (0.1) | 1.6 (0.0) | 1.5 (0.1) | 2.7 (0.5) |
| | glycoprotein 1) | | | | |
| U47008 | NGFI-A binding protein 1 (EGR-1 binding protein 1) | 10.2 (1.8) | 1.9 (0.3) | 1.3 (0.6) | 1.5 (0.2) |
| U47543 | NGFI-A binding protein 2 (EGR-1 binding protein 2) | 1.8 (0.3) | 1.7 (0.0) | 2.0 (0.3) | 1.6 (0.1) |
| U56773 | Interleukin-1 receptor-associated kinase 1 (IRAK-1) | 3.2 (0.2) | 2.0 (0.1) | 1.7 (0.1) | 1.4 (0.1) |
| | (IRAK) pelle-like protein kinase) (MPLK) | | | | |
| X06407 | Translationally controlled tumor protein (TCTP) (P23) | 1.3 (0.1) | 1.4 (0.1) | 1.3 (0.1) | 1.4 (0.2) |
| | (21 KD polypeptide) P21) (lens epithelial protein) | | | | |
| X54352 | F-BOX/WD-repeat protein 2 (MD6 protein) | 1.7 (0.1) | 1.6 (0.1) | 1.4 (0.1) | 1.4 (0.0) |
| X84037 | Selectin, endothelial cell, ligand | 1.8 (0.1) | 2.0 (0.1) | 2.1 (0.2) | 1.5 (0.2) |
| Y00746 | Retinal rod rhodopsin-sensitive CGMP 3′,5′-cyclic | 1.4 (0.1) | 2.3 (0.9) | 3.6 (1.0) | 3.7 (1.6) |
| | phosphodiesterase gamma-subunit (GMP-PDE gamma) | | | | |
| Y07688 | Nuclear factor 1/X (NFI-X) (NF-I/X) (CCAAT-box | 1.6 (0.2) | 3.5 (0.1) | 3.8 (0.3) | 1.5 (0.3) |
| | binding transcription factor) (CTF) (TGGCA-binding | | | | |
| | protein) | | | | |
| Z19581 | Seven in absentia 2 (siah2) | 1.6 (0.1) | 1.7 (0.1) | 2.8 (0.4) | 1.8 (0.2) |
| Z46966 | Islet mitochondrial antigen, 38 kD; imogen 44 | 1.3 (0.1) | 1.3 (0.1) | 1.3 (0.1) | 1.3 (0.1) |

Down with CR in Four Postmitotic Tissues

| | | | | | |
|---|---|---|---|---|---|
| AF003695 | Hypoxia inducible factor 1, alpha subunit | −1.5 (0.1) | −1.5 (0.0) | −1.9 (0.3) | −1.4 (0.1) |
| AF020772 | Importin alpha-3 subunit (karyopherin alpha-3 subunit) | −2.6 (0.1) | −3.1 (0.4) | −3.5 (0.8) | −3.6 (0.4) |
| C76063 | Unknown | −2.7 (0.4) | −3.3 (0.2) | −3.3 (1.4) | −4.9 (0.7) |
| C79663 | Unknown (no good homology) | −9.4 (1.4) | −4.3 (0.9) | −5.2 (2.9) | −6.2 (1.6) |
| D10715 | Developmentally regulated GTP-binding protein 1 (DGR | −2.3 (0.1) | −2.0 (0.1) | −1.5 (0.1) | −1.3 (0.1) |
| | 1) (NEDD3 protein) | | | | |
| D12713 | Protein transport protein SEC23A | −4.3 (1.6) | −4.2 (0.9) | −4.2 (1.0) | −5.4 (1.1) |
| D67076 | ADAM-TS 1 precursor (a disintegrin and | −1.3 (0.1) | −5.3 (1.3) | −4.5 (2.8) | −4.4 (1.0) |
| | metalloproteinase with thrombospondin motifs 1) | | | | |
| | (ADAMTS-1) (ADAM-TS1) | | | | |
| D86344 | Programmed cell death 4 | −1.9 (0.2) | −1.8 (0.1) | −3.4 (1.3) | −3.1 (0.8) |
| L10244 | Diamine acetyltransferase (spermidine/spermine N1- | −1.4 (0.1) | −1.7 (0.0) | −3.0 (1.5) | −2.3 (0.3) |
| | acetyltransferase) (SSAT) (putrescine acetyltransferase) | | | | |
| L18888 | Calnexin | −2.5 (0.3) | −1.4 (0.1) | −2.1 (0.5) | −1.4 (0.1) |
| M57966 | Hepatocyte nuclear factor 1-alpha (HNF-1A) (liver | −1.7 (0.1) | −1.6 (0.1) | −1.5 (0.2) | −1.7 (0.3) |
| | specific transcription factor LF-B1) (LFB1) | | | | |
| M58564 | Butyrate response factor 2 (TIS11D protein) | −10.8 (7.0) | −2.6 (0.1) | −5.2 (0.7) | −3.7 (2.1) |
| U19463 | Tumor necrosis factor, alpha-induced protein 3 (putative | −4.6 (1.2) | −4.8 (0.6) | −3.7 (1.9) | −2.9 (1.0) |
| | DNA binding protein A20) (zinc finger protein a20) | | | | |
| U25844 | Serine protease inhibitor 3 (spi3) | −1.5 (0.1) | −1.5 (0.1) | −1.6 (0.2) | −1.3 (0.1) |
| U27830 | Extendin (stress response) | −1.7 (0.1) | −2.4 (0.4) | −1.6 (0.2) | −3.4 (0.8) |
| U35623 | EAT/MCL-1 | −1.4 (0.1) | −1.4 (0.0) | −2.3 (0.4) | −1.3 (0.1) |
| U43892 | ATP-binding cassette, sub-family B, member 7, | −1.9 (0.2) | −1.8 (0.3) | −2.0 (0.3) | −2.5 (0.3) |
| | mitochondrial(ATP-binding cassette transporter 7) (ABC) | | | | |
| | transporter 7 protein) | | | | |
| U51204 | APC-binding protein EB2 | −1.6 (0.2) | −2.4 (0.1) | −1.9 (0.4) | −3.9 (0.9) |
| U75321 | Potential phospholipid-transporting atpase IA (chromaffin | −3.0 (0.5) | −2.4 (0.2) | −5.2 (2.4) | −1.6 (0.1) |
| | granule ATPase III) | | | | |
| U84207 | Cholinephosphate cytidylyltransferase A | −4.2 (0.4) | −2.0 (0.3) | −1.5 (0.1) | −1.8 (0.3) |
| | (Phosphorylcholine Transferase A) (CTP: | | | | |
| | Phosphocholine Cytidylyltransferase A) (CT A) (CTT A) | | | | |
| | (CTT-alpha) | | | | |
| X52914 | H2-D (locus 4) | 13.9 (7.9) | −4.6 (0.5) | −7.2 (3.3) | 28.6 (5.0) |
| X54424 | Adapter-related protein complex 1 gamma 1 subunit | −2.7 (0.4) | −3.5 (0.2) | −1.8 (0.7) | −2.1 (0.9) |
| | (gamma-adaptin) (golgi adaptor HA1/AP1 adaptin | | | | |
| | gamma subunit) (clathrin assembly protein complex 1 | | | | |
| | gamma large chain) | | | | |
| X75926 | ATP-binding cassette, sub-family A, member 1 (ATP- | −5.1 (0.6) | −1.8 (0.7) | −2.4 (0.5) | −3.6 (0.5) |
| | binding cassette transporter 1) (ATP-binding cassette 1) | | | | |
| | (ABC-1) | | | | |
| X99921 | S100 calcium-binding protein A13 | −10.3 (1.0) | −1.5 (0.0) | −1.3 (0.1) | −1.3 (0.01) |
| Z47088 | Cyclin A/CDK2-associated protein P19 (RNA | −1.5 (0.1) | −3.3 (0.1) | −2.5 (0.4) | −1.6 (0.2) |
| | polymerase II elongation factor-like protein) (organ of | | | | |
| | corti protein 2) (OCP-II protein) (OCP-2) (transcription | | | | |
| | elongation factor B) (SIII) | | | | |

We now describe the functions of the genes identified as transcriptional biomarkers of CR shared among multiple organs. Also, appended are Figures showing fold changes and signal intensities for these genes in the tissues showing shared expression changes.

B. Genes altered in expression in all six tissues. Three genes, RNA Polymerase 140Kd subunit (ORF M21050), an unknown gene (R74626) and Noggin precursor (U79163) were induced by CR by 1-5-fold (500%) or more in all tissues, whereas five genes, Complement C1qB (M22531), Selenophosphate synthetase 2 (U43285), Peptidylglycine alpha-amidating monooxygenase (U79523), teg271 (X81059), and Rab5b (X84239) were decreased in expression by 50% or more in all tissues studied. Relevant information regarding possible functions is provided if available as extracted from GenBank and PubMed.

1. Genes Increased in Expression in Six Tissues

RNA polymerase 140Kd subunit (ORF M21050) is a DNA dependent RNA polymerase that catalyzes the transcription of DNA into RNA for ribosomal RNA precursors (Paule and White, 2000). The transcription of RNA polymerase I has been reported to decrease with age in Droshophila leading to the suggestion that this change could contribute to age-associated decreases in protein synthesis (Shikama and Brack, 1996). A decrease in protein synthesis is one of the most commonly observed biochemical changes during aging (Rattan, 1996) and there is good evidence to suggest that CR increases rates of protein synthesis (Weindruch and Walford, 1988). Therefore, it is possible that the increased expression of the 40 Kd subunit of RNA Polymerase I may represent a change of fundamental importance in the ability of CR to retard the aging process.

Unknown (R74626): No homology >30% was found in a BLAST search.

Noggin precursor (U79163): The secreted polypeptide noggin (encoded by the Nog gene) binds and inactivates members of the transforming growth factor beta superfamily of signaling proteins (TGFbeta-FMs), such as BMP4. By diffusing through extracellular matrices more efficiently than TGFbeta-FMs, noggin may have a principal role in creating morphogenic gradients. During mouse embryogenesis, Nog is expressed at multiple sites, including developing bones. Nog−/−mice die at birth from multiple defects that include bony fusion of the appendicular skeleton. Recently, it has been demonstrated that noggin is required for mouse forebrain development (Bachiller, et al., 2000). Although little else is known about the function of noggin in mammals, the widespread upregulation by CR of a gene encoding of a molecule which induces neuronal tissues (Gong, et al., 1999) is intriguing.

2. Genes Decreased in Expression in Six Tissues

Complement C1qB: This is a component of the complement cascade which is an evolutionarily conserved part of the innate immune system. The subcomponent of complement C1, C1q, mediates complement activation via the classical pathway and therefore may play an important role in the inflammatory processes in which complement activation is involved. Production of complement proteins in the brain contributes to neuronal damage associated with stroke (Huang, et al., 1999) and has been observed in the striatum of old rats (Pasinetti, et al., 1999).

Selenophosphate synthetase 2 (U43285): Synthesis of monoselenophosphate, the selenium donor required for the synthesis of selenocysteine (Sec), is catalyzed by the enzyme selenophosphate synthetase (SPS). It synthesizes selenophosphate from selenide and ATP. Expression of individual eukaryotic selenoproteins exhibits high tissue specificity, depends on selenium availability, in some cases is regulated by hormones, and if impaired contributes to several pathological conditions (Kohrl, et al., 2000). A decreased expression of the SPS 2 gene may derive from a decreased state of oxidative stress in mice on CR.

Peptidylglycine alpha-amidating monooxygenase (U79523, PAM): PAM catalyzes the copper-, ascorbate-, and O(2)-dependent cleavage of C-terminal glycine-extended peptides and N-acylglycines to the corresponding amides and glyoxylate. The alpha-amidated peptides and the long-chain acylamides are hormones in humans and other mammals.

teg271 (X81059) is a gene expressed early in mouse spermatogenesis. Little is known about this gene and the protein that it encodes.

Rab5b (X84239) encodes a protein that is likely to be involved in vesicular traffic. It has similarity to RAS proteins and belongs to the RAB subfamily. Interestingly, Rab5B in the total membrane fraction of human skeletal muscle was 2.1- to 3.6-fold higher in insulin resistant subjects than in insulin sensitive individuals (Bao, et al., 1998). The decrease in Rab5b expression induced by CR may have some relationship to the increased insulin sensitivity observed in rodents and primates subjected to CR.

C. Seventeen Genes Upregulated by CR in 5 out of 6 Tissues.

1. Upregulated in all but Gastrocnemius

m-calpain (D38117) is a calcium-activated, non-lysosomal thiol-protease and is similar to EF-Hand calcium binding proteins. It was upregulated by CR in all tissues but the gastrocnemius. Conventional calpains are ubiquitous calcium-regulated cysteine proteases that have been implicated in cytoskeletal organization, cell proliferation, apoptosis, cell motility, and hemostasis. There are two forms of conventional calpains: the mu-calpain, or calpain I, which requires micromolar calcium for half-maximal activation, and the m-calpain, or calpain II, which functions at millimolar calcium concentrations. It was recently reported that m-calpain may be responsible for cleaving procaspase-12, a caspase localized in the ER, to generate active caspase-12 (Nakagawa and Yuan, 2000). In addition, calpain may be responsible for cleaving the loop region in Bcl-xL and, therefore, turning an antiapoptotic molecule into a proapoptotic molecule.

Connective tissue growth factor precursor (CTGF)/hypertrophic chondrocyte-specific gene product 24 (CTGF/Hcs24) (M70642): CTGF/Hcs24 is a widely studied, multifunctional growth factor for fibroblasts, chondrocytes, and vascular endothelial cells (reviewed by Moussad and Brigstock, 2000). CTGF is a member of the recently described CCN gene family which contains CTGF itself, cyr6l, nov, elm1, Cop1, and WISP-3. CTGF is transcriptionally activated by several factors, although its stimulation by transforming growth factor beta (TGF-beta) has attracted considerable attention. CTGF acts to promote fibroblast proliferation, migration, adhesion, and extracellular matrix formation, and its overproduction is proposed to play a major role in pathways that lead to fibrosis, especially those that are TGF-beta-dependent. This includes fibrosis of major organs, fibroproliferative diseases, and scarring. CTGF also appears to play a role in the extracellular matrix remodeling that occurs in normal physiological processes such as embryogenesis, implantation, and wound healing. However, recent advances have shown that CTGF is involved in diverse autocrine or paracrine actions in several other cell types such as vascular endothelial cells, epithelial cells, neuronal cells, vascular smooth muscle cells, and cells of supportive skeletal tissues. Moreover, in some circumstances CTGF has negative effects on cell growth in that it can be antimitotic and apoptotic. In light of these discoveries, CTGF has been implicated in a diverse variety of processes that include neovascularization, transdifferentiation, neuronal scarring, atherosclerosis, cartilage differentiation, and endochondral ossification. Also, there are reports (Hishikawa, et al., 1999) of CTGF inducing apoptosis.

Tuberin (Tuberous Sclerosis 2 Homolog Protein) U37775

Two genes, TSC1 and TSC2, have been shown to be responsible for tuberous sclerosis (TSC). The detection of loss of heterozygosity of TSC1 or TSC2 in hamartomas, the growths characteristically occurring in TSC patients, suggested a tumor suppressor function for their gene products hamartin and tuberin (Hengstschlager, et al., 2000). Studies analyzing ectopically modulated expression of TSC2 in human and rodent cells together with the finding that a homolog of TSC2 regulates the Drosophila cell cycle suggest that TSC is a disease of proliferation/cell cycle control and that these genes are involved in these processes.

Protein tyrosine phosphatase IVA1 (U84411) is poorly characterized.

2. Upregulated in all but Heart

Presynaptic protein SAP102 (D87117) interacts with the cytoplasmic tail of the NMDA receptor subunit NR2B. SAP102 is a membrane-associated guanylate kinase protein which interacts with its N-terminal segments designated the PDZ domains and acts to cluster these receptors at the target site of the cell membrane. SAP102 is thought to be a neuronal and endocrine tissue-specific MAGUK family protein expressed in both dendrites and cell bodies in neuronal cells (Fujita and Kurachi, 2000).

Carbonyl reductase (U31996) belongs to the family of short-chain dehydrogenases/reductases (reviewed by Forrest, et al., 2000). Carbonyl reductases (CBRs) are NADPH-dependent, mostly monomeric, cytosolic enzymes with broad substrate specificity for many endogenous and xenobiotic carbonyl compounds. Like isocitrate dehydrogenase 2, it too generates NADPH. Emerging data on CBRs indicate the potential involvement of CBRs in a variety of cellular and molecular reactions associated with drug metabolism, detoxification, drug resistance, mutagenesis, and carcinogenesis.

Isocitrate dehydrogenase 2 (U51167) plays a role in intermediary metabolism and energy production. The reaction produces NADPH, which is a critically important molecule to support the reducing functions of several antioxidant pathways. Interestingly, yeast isocitrate dehydrogenase (Idh) binds specifically and with high affinity to the 5′-untranslated leader sequences of mitochondrial mRNAs in vitro and may play a role in the regulation of mitochondrial translation (Elzinga, et al., 2000).

3. Upregulated in all but Kidney

Pink-eyed dilution (M97900): Recessive mutations of the mouse p (pink-eyed dilution) gene lead to hypopigmentation of the eyes, skin, and fur (reviewed in Brilliant, 2000). Mice lacking a functional p protein have pink eyes and light gray fur (if non-agouti) or cream-colored fur (if agouti). The human orthologue is the P protein. Humans lacking a functional P protein have oculocutaneous albinism type 2 (OCA2). Melanocytes from p-deficient mice or OCA2 individuals contain small, minimally pigmented melanosomes. The mouse and human proteins are predicted to have 12 membrane spanning domains and possess significant sequence homology to a number of membrane transport proteins, some of which are involved in the transport of anions. The p protein has been localized to the melanosome membrane. Recently, it has been shown that melanosomes from p protein-deficient melanocytes have an abnormal pH. Melanosomes in cultured melanocytes derived from wild-type mice are typically acidic, whereas melanosomes from p protein-deficient mice are non-acidic. Melanosomes and related endosome-derived organelles (i.e., lysosomes) are thought to have an adenosine triphosphate (ATP)-driven proton pump that helps to generate an acidic lumen. To compensate for the charge of these protons, anions must also be transported to the lumen of the melanosome. In light of these observations, a model of p protein function is presented in which the p protein, together with the ATP-driven proton pump, regulates the pH of the melanosome. These findings suggest that the expression of the pink-eyed dilution gene may be regulated by ATP levels, providing a potential explanation for the decreased expression of this gene in multiple organs by caloric restriction.

Serum paraoxonase (PON 1) (U32684) hydrolyzes the toxic metabolites of a variety of organophosphorous insecticides, and therefore may function in detoxification. This widely studied enzyme is a Ca²⁺-dependent 45-kDa glycoprotein that is associated with high density lipoprotein (HDL). There is considerable evidence that the antioxidant activity of high density lipoprotein (HDL) is largely due to the paraoxonase-1 (PON1) located on it (Durrington, et al., 2001). Experiments with transgenic PON1 knockout mice indicate the potential for PON1 to protect against atherogenesis. Also, there is evidence that the genetic polymorphisms of PON1 least able to protect LDL against lipid peroxidation are over-represented in coronary heart disease, particularly in association with diabetes.

Vascular endothelial growth factors-B (VEGF-B) (U43836) is a growth factor for endothelial cells that can form heterodimers with VEGF. VEGFs constitute a group of structurally and functionally related growth factors that modulate many important physiological functions of endothelial cells (Li and Eriksson, 2001). Currently, five different mammalian VEGFs have been identified and they all show unique temporal and spatial expression patterns, receptor specificity and function. The VEGFs may play pivotal roles in regulation of capillary growth in normal and pathological conditions in adults, and in the maintenance of the normal vasculature. Although the specific functions of VEGF-B are poorly understood, a recent analysis of mice with a targeted deletion of the VEGF-B gene has revealed a defect in heart development and function consistent with an important role in vascularization of the myocardium (Bellomo, et al., 2000).

Histidine triad nucleotide-binding protein (protein kinase C inhibitor 1) (PKCI-1) (U60001) does not function as an inhibitor of PKC, but rather acts as an enzyme in a yet to be identified pathway (Klein, et al., 1998). It appears to be an intracellular receptor for purine mononucleotides which possesses an enzymatic activity cleaving ADP into AMP and inorganic phosphate. Thus, the molecule appears to be related to bioenergetics.

Brain protein 1 (X61450) is not described in any scientific publication that we could locate and, accordingly, is of unknown function.

4. Upregulated in all but Liver

Sox 17 (D49473) is a probable transcriptional activator in the premeiotic germ cells. It binds to sequences 5′-MCAAT-3′ or 5′-MCAAAG-3′. The Sox gene family (Sry like HMG box gene) is characterized by a conserved DNA sequence encoding a domain of approximately 80 amino acids which is responsible for sequence specific DNA binding.

60S ribosomal protein L29 (L08651). This gene encodes a protein that belongs to the L29E family of 60S ribosomal proteins; thus, it is involved in protein synthesis.

60S ribosomal protein L13 (U28917). This gene encodes a protein that belongs to the L13E family of 60S ribosomal proteins; thus, it is involved in protein synthesis. L13 is one of a group of ribosomal proteins may function as cell cycle checkpoints and comprise a new family of cell proliferation regulators (Chen and loannou, 1999). For example, inhibition of expression of L13 induces apoptosis in target cells, suggesting that this protein is necessary for cell survival.

Lisch7 (U49507) is a poorly characterized transcriptional factor (Steingrimsson, et al., 1995).

Gas 6 (X59846) is being actively studied and is involved in cell growth arrest. GAS6 is a ligand for the AxI (Ufo/Ark), Sky (Dtk/Tyro3/Rse/Brt/Tif), and Mer (Eyk) family of tyrosine kinase receptors and binds to these receptors via tandem G domains at its C terminus (Dormady, et al., 2000). After translation, GAS6 moves to the lumen of the endoplasmic reticulum, where it is extensively gamma-carboxylated. The carboxylation process is vitamin K dependent, and current evidence suggests that GAS6 must be gamma-carboxylated to bind and activate any of the cognate tyrosine kinase receptors. The Gas6/AxI system is believed to play critical regulatory roles in diverse systems including vascular (Melaragno, et al., 1999) and neuronal (Tsaioun, 1999) cell function.

D. Four Genes Downregulated in Five of the Six Tissues.

1. Downregulated in all but Gastrocnemius

H-2 Class II histocompatibility Antigen, E-B Beta Chain Precursor (X00958) is an immune response gene of the major histocompatibility complex (MHC). Class II proteins are expressed on lymphocytes of various types.

2. Downregulated in all but Heart

Mitogen-regulated protein 2 (Mrp2) (Proliferin 2) (K0325) is a growth factor that belongs to the Somatotropin/prolactin family. Mitogen-regulated proteins are expressed at high levels during midgestation when they are thought to induce angiogenesis and uterine growth. There are between four and six mrp/plf genes. Genes of the Proliferin family are induced by oxidative stress (Parfeft and Pilon R, 1995).

Hypothetical protein (B2 element) (Z48238). This gene is a homolog (73% homology) to one that encodes an uncharacterized protein.

3. Downregulated in all but Kidney

Gamma-aminobutyric-acid receptor delta subunit precursor (GABA(A) receptor) (M60596) is the major inhibitory neurotransmitter in the brain and, accordingly, is the subject of intensive study. It is an integral membrane protein which mediates neural inhibition by binding to the GABA/benzodiazepine receptor and opening a chloride channel.

E. Thirty Genes Upregulated in the Four Post-Mitotic Tissues Examined (Gastrocnemius, Heart, Cerebellum and Neocortex).

AA117417 Is a gene of unknown function (no significant homology to the database).

Phosphomannomutase 1 (PMM 1) (AA117417) is involved in the synthesis of the GDP-mannose and Dolichol-phosphate-mannose required for a number of critical mannosyl transfer reactions. It is thought to function in glycosylation and the early steps of mannosylation.

Putative oral cancer suppresssor (deleted in oral cancer-1) (DOC-1) (AF011644) is a putative tumor suppressor gene isolated and identified from the hamster oral cancer model. There is evidence that doc-1 induces apoptosis in malignant hamster oral keratinocytes (Cwikla, et al., 2000). Doc-1 is an evolutionarily conserved gene exhibiting loss of heterozygosity and marked reduction in expression in malignant hamster oral keratinocytes (Todd, et al., 1995). The full-length doc-1 cDNA encodes an 87 amino acid product that shows a significant homology to one of the seven novel genes induced in mouse fibroblasts by TNF-alpha.

Complement component 1 Q subcomponent binding protein (AJ001101) binds to the globular heads of C1Q thus inhibiting C1 activation. It has a mitochondrial localization (but not exclusively) (Soltys, et al., 2000). gC1qBP is a novel cell protein which was also found to interact with the globular heads of high molecular weight kininogen, factor XII and the heparin-binding, multimeric form of vitronectin. The protein sequence shows no homology to any protein family.

40S ribosomal protein S17 (C79471) belongs to the S17E family of ribosomal proteins. S17 is a primary rRNA-binding protein, which has been implicated in ribosome assembly and translational fidelity.

Coproporphyrinogen III oxidase (coproporphyrinogenase) (coprogen oxidase) (D16333) is a mitochondrial enzyme which catalyzes the sixth step in heme biosynthesis. Using O₂, it converts coproporphyrinogen III (coprogen) to protoporphyrinogen IX (protogen) and 2CO₂.

Farnesyltransferase alpha subunit (CAAX farnesyltransferase alpha subunit) (RAS proteins prenyltransferase alpha (FTASE-alpha) (D49744) catalyzes the transfer of a farnesyl moiety from farnesyl pyrophosphate to a cysteine at the fourth position from the C-terminus of several proteins. Recent observations have linked the protein encoded by this gene to apoptosis: farnesyltransferase/geranylgeranyl-transferase (FTase/GGTase)-alpha, a common subunit of FTase (alpha/beta(FTase)) and GGTase I (alpha/beta(GGTase)), was cleaved by caspase-3 during apoptosis (Kim, et al., 2000). Also of major interest is the observation that insulin activates farnesyltransferase (FTase) and augments the amounts of farnesylated p21 (Goalstone and Draznin, 1996). Recent data suggest that insulin signaling from its receptor to the prenyltransferases FTase and GGTase I is mediated by the Shc pathway, but not the IRS-1/phosphatidylinositol 3-kinase pathway (Goalstone, et al., 2001). Shc-mediated insulin signaling to MAPK may be necessary (but not sufficient) for activation of prenyltransferase activity. It is noteworthy that our data suggest that this gene is highly expressed in all four postmitotic tissues with remarkably little variation among the individual mice in the CR group (higher expression) and the control group (lower expression).

Homeobox protein SIX5 (D83146). Previously known as myotonic dystrophy associated homeodomain protein—DMAHP, it is a member of the SIX [sine oculis homeobox (Drosophila) homologue] gene family which encodes proteins containing a SIX domain adjacent to a homeo-domain. Mice deficient in Six5 develop cataracts (Klesert, et al., 2000).

High-sulfur keratin protein (D86424) has unknown function.

Adrenodoxin, mitochondrial precursor (L29123) transfers electrons from adrenodoxin reductase to the cholesterol side chain cleavage cytochrome P450 (reviewed by Grinberg, et al., 2000). It is located in the mitochondrial matrix. Adrenodoxin is an iron-sulfur protein that belongs to the broad family of the [2Fe-2S]-type ferredoxins found in plants, animals and bacteria. Its primary function as a soluble electron carrier between the NADPH-dependent adrenodoxin reductase and several cytochromes P450 makes it an irreplaceable component of the steroid hormones biosynthesis in the adrenal mitochondria of vertebrates.

Ankyrin 3, (L40632) is a protein linker between the integral membrane proteins and spectrin-based cytoskeleton (reviewed in Rubtsov and Lopina, 2000). Ankyrins participate in signal transduction and in assembly of integral membrane proteins in specialized membrane domains. Ankyrin-3 (also called ankyrin(G)), is widely distributed, especially in epithelial tissues, muscle, and neuronal axons (Peters, et al., 1995).

House-keeping protein I (M74555) has no known function.

Follistatin-related protein 1 (TGF-beta-inducible protein TSC-36) (M91380) is thought to modulate the action of some growth factors on cell proliferation and differentiation. TSC-36 (TGF-betal-stimulated clone 36) is a TGF-betal inducible gene whose product is an extracellular glycoprotein that contains a single follistatin module. TSC-36's physiological function is unknown. The protein encoded by this gene has largely been investigated in the context of cancer. For example, TSC-36 caused growth inhibition in human lung cancer cells (Sumitomo, et al., 2000).

Glycosylation-dependent cell adhesion molecule 1 (GLYCAM-1) (M93428) encodes an adhesion molecule that accomplishes cell binding by presenting carbohydrates to the lectin domain of L-selectin. It is a mucin-like endothelial glycoprotein. However, it is now clear that it is expressed elsewhere such as in cells of the cochlea (Kanoh, et al., 1999).

IkB-beta (U19799) is an inhibitor of Nuclear factor-kappaB (NF-kappa B). NF-kappa B is a pleiotropic oxidant-sensitive transcription factor that is present in the cytosol in an inactive form complexed to an inhibitory kappaB (I kappa B) monomer. Various stimuli, including ischemia, hypoxia, free radicals, cytokines, and lipopolysaccharide (LPS), activate NF-kappa B by inducing phosphorylation of I kappa B. Recent evidence has linked this system to mitochondrial apoptosis pathways. For example, IkappaB Alpha, another NF-kappaB inhibitory subunit, interacts with ANT, the mitochondrial ATP/ADP translocator (Bottero, et al., 2001). Further, IkB-a/NF-kB appeared to be released from mitochondria upon induction of apoptosis.

Interestingly, the gene encoding IkB-beta was highly expressed in the four postmitotic tissues studied (Signal Intensities 1391 to 4362), while it was very weakly expressed in kidney and liver (SI=−669 to −1224) irrespective of diet group.

Kruppel-like factor 4 (Epithelial zinc-finger protein EZF) (U20344) acts as a transcriptional factor that binds to the CACCC core sequence, and may be involved in the differentiation of epithelial cells. In humans, EZF is expressed in vascular endothelial cells and contains transcriptional activation and repression domains (Yet, et al., 1998).

Phosphoserine/threonine/tyrosine interaction protein (STYX) (U34973) encodes a phosphoserine/threonine/tyrosine-binding protein. Dual-specificity protein-tyrosine phosphatases (dsPTPases) have been implicated in the inactivation of mitogen-activated protein kinases (MAPKs). STYX is a unique modular domain found within proteins implicated in mediating the effects of tyrosine phosphorylation in vivo (reviewed by Wishart and Dixon, 1998). Individual STYX domains are not catalytically active; however, they resemble protein tyrosine phosphatase (PTP) domains and, like PTPs, contain core sequences that recognize phosphorylated substrates. Thus, the STYX domain adds to the repertoire of modular domains that can mediate intracellular signaling in response to protein phosphorylation.

Nuclear receptor co-repressor 1 (N-COR1) (N-COR) (retinoid X receptor interacting protein 13) (RIP13). U35312 retinoid X receptors (RXRs) are involved in a number of signaling pathways as heterodimeric partners of numerous nuclear receptors. RIP13 mediates the transcriptional repression activity of some nuclear receptors by promoting chromatin condensation, thus preventing access of transcriptional factors. It forms a large corepressor complex that contains sin3A/B and histone deacetylases HDAC1 and HDAC2. This complex associates with the thyroid and retinoic acid receptors in the absence of ligand. The linkage with the thyroid axis is particularly intriguing in view of the hypometabolic state induced by CR. This study of RXRs and associated molecules is an impressively active area of inquiry and worthy of more thought/investigation from a gerontological perspective.

Puromycin-sensitive aminopeptidase (Psa) (U35646) has broad substrate specificity to several peptides. It is involved in proteolytic events which are essential for cell growth and viability. It also may act as a regulator of neuropeptide activity and displays highest expression in the brain (especially in the striatum and hippocampus). Studies of a mouse strain which has this gene disrupted indicate that Psa is required for normal growth and the behavior associated with anxiety and pain (Osada, et al., 1999).

Dystroqlycan precursor (dystrotrophin-associated glycoprotein 1) (U43512) forms part of the dystrophin-associated protein complex, which may link the cytoskeleton to the extracellular matrix. The precursor contains both alpha-dystroglycan (alpha-DG) and beta-DG. Alpha-DG functions as a laminin receptor and has an extracellular localization. Beta-DG is a type-1 membrane protein. In the heart, sarcolemma integrity is stabilized by the dystrophin-associated glycoprotein complex that connects actin and laminin-2 in contractile machinery and the extracellular matrix, respectively. The importance of the proteins encoded by this gene to the aging process are clearly illustrated by studies in rat hearts. Interruption of the dystrophin-dependent connections by the primary gene defect or acquired pathological burden can cause cardiac failure. Xi, et al. (2000) investigated whether dystrophin is disrupted in acute myocardial injury after isoproterenol overload and examined its relation to myocardial cell apoptosis in rats. They observed that beta-adrenergic stimulation induces dystrophin breakdown followed by apoptosis. Perhaps the 2.7-fold CR-induced overexpression of this highly expressed gene in the heart (Signal Intensity of 7802 for Control vs. 19,829 for CR) provides a mechanism to oppose myocardial cell apoptosis. Similarly, mutations of this gene cause skeletal muscle diseases including some types of muscular dystrophy.

NGF-1 Binding Protein 1 (EGR-1 BP1) (U47008) and NGF-1 Binding Protein 2 (EGR-1 BP2) (U47543) act as transcriptional repressors for the Zinc finger transcription factors EGR1 (also called Krox24) and EGR2. The co-upregulation of these two genes in the four postmitotic tissues studied is remarkable. Egr-1 is an immediate early gene that couples short-term changes in the extracellular milieu to long-term changes in gene expression. Under in vitro conditions, the Egr-1 gene is expressed in many cell types and is induced by a wide variety of extracellular signals. The mechanisms by which the Egr-1 gene is regulated in vivo remain poorly understood. The coordinated induction of EGR-1 BP1 and EGR-1 BP2 may represent early transcriptional changes caused by CR which precede and underlie long-term alterations in gene expression in this model of aging retardation.

Interleukin-1 receptor-associated kinase 1 (IRAK-1) (IRAK) pelle-like protein kinase) (MPLK) (U56773) is involved in IL-1 pathway. This kinase associates with the IL-1 receptor IL1-R-1. This association is rapid and IL-1 dependent. It is a member of the Toll-like receptors (TLRs), which are involved in innate immunity (Muzio, et al., 2000). Toll is a Drosophila gene essential for ontogenesis and anti-microbial resistance. Several orthologues of Toll have been identified and cloned in vertebrates. TLRs are characterized structurally by a cytoplasmic Toll/interleukin-1 receptor (TIR) domain and by extracellular leucine-rich repeats.

Translationally controlled tumor protein (tctp) (P23) (21 KD polypeptide) (P21) (lens epithelial protein) (X06407) is a growth-related protein, which is regulated at the translational level. It is present in mammals, higher plants and Saccharomyces cerevisiae. Tctp is found in several healthy and tumor cells including erythrocytes, hepatocytes, macrophages, platelets, keratinocytes, erythroleukemia cells, gliomas, melanomas, hepatoblastomas, and lymphomas (Sanchez, et al., 1997). The high degree of homology from plants to man and its expression in many tissues suggests that tctp may have a cell housekeeping function. This idea is supported by the extremely high signal intensities observed in our study, which ranged from 30,000 to 80,000 among the six tissues assayed. The expression of translationally controlled tumor protein is regulated by calcium at both the transcriptional and post-transcriptional level (Xu, et al., 1999).

F-Box/WD-Repeat Protein 2 (MD6 PROTEIN) (X54352) probably recognizes and binds some phosphorylated proteins and promotes their ubiquitination and degradation. The F-box is a protein motif of approximately 50 amino acids that functions as a site of protein-protein interaction (reviewed by Kipreos and Pagano, 2000). F-box proteins were first characterized as components of SCF ubiquitin-ligase complexes (named after their main components, Skp I, Cullin, and an F-box protein), in which they bind substrates for ubiquitin-mediated proteolysis. The F-box motif links the F-box protein to other components of the SCF complex by binding the core SCF component Skp I. F-box proteins have more recently been discovered to function in non-SCF protein complexes in a variety of cellular functions. There are 11 F-box proteins in budding yeast, 326 predicted in C. elegans, 22 in Drosophila, and at least 38 in humans. F-box proteins often include additional carboxy-terminal motifs capable of protein-protein interaction; the most common secondary motifs in yeast and human F-box proteins are WD repeats and leucine-rich repeats, both of which have been found to bind phosphorylated substrates to the SCF complex. The majority of F-box proteins have other associated motifs, and the functions of most of these proteins have not yet been defined.

Miura, et al. (1999) isolated a cDNA encoding the mouse F-box/WD-Repeat protein 2 (also known as Fwd2 and MD6). Fwd2 cDNA contains 1890 bp with a 1362-bp open reading frame and encodes an ˜51.5-kDa protein. They observed that Fwd2 is expressed predominantly in liver and, to a lesser extent, in the testis, lung, heart, and skeletal muscle. Immunofluorescence staining for Fwd2 protein shows a pattern with the cytoplasm. A coimmunoprecipitation assay has revealed the in vivo interaction between Skp1 and Fwd2 through the F-box domain. Fwd2 also interacts with Cull through Skp1, suggesting that Skp1, Cull, and the F-box protein Fwd2 form an SCF complex (SCF(Fwd2)). These data suggest that Fwd2 is an F-box protein that constitutes an SCF ubiquitin ligase complex and that it plays a critical role in the ubiquitin-dependent degradation of proteins.

Selectin (endothelial cell type, E-selectin) (X84037). Selectins are carbohydrate-binding adhesive proteins of three types. The E, L and P forms of members of this family bind specifically to carbohydrates on endothelium, lymph node vessels and activated platelets, respectively. Each contains a conserved 120-residue carbohydrate-recognition domain (CRD) that complexes Ca++ together with the specific carbohydrate. The upregulation of this gene by CR is curious given that this E-selectin is increased in expression in a variety of inflammatory states (Gonzalez-Amaro and Sanchez-Madrid, 1999) versus the broad set of data supporting the idea that CR downregulates basal states of inflammation (Weindruch and Walford, 1988; Lee, et al., 2000). It is interesting to note that the expression of E-selectin on glial cells and activated astrocytes has recently been observed (Lee and Benveniste, 1999) and is of unknown functional significance.

Retinal rod rhodopsin-sensitive CGMP 3′,5′-cyclic phosrhodiesterase gamma-subunit (GMP-PDE gamma) (Y00746) participates in processes of transmission and amplification of the visual signal. CGMP-PDEs are the effector molecules in G-protein-mediated phototransduction in rods and cones. The reaction is to convert cGMP into GMP. The enzyme is oligomer (=two catalytic chains [alpha, beta], and inhibitory chain [gamma] and the delta chain). Thus, CR upregulates the inhibitory chain.

Nuclear factor 1/X (NFI-X) (NF-I/X) (CCAAT-box binding transcription factor) (CTF) (TGGCA-binding protein) (Y07688). CTF/NF-1 is a transcriptional activator. It appears to be particularly sensitive to oxidative stress (Barouki and Morel, 2001) and other cellular stresses including inflammation, glutathione depletion, heat and osmotic shocks, and chemical stress (Morel, et al., 2000). For example, beyond Cytochrome P450 1A1's (CYP1A1) usual role in detoxification of polycyclic aromatic compounds, the activity of this enzyme can be deleterious since it can generate mutagenic metabolites and oxidative stress. Accordingly, several feedback loops control the activation of this gene and the subsequent potential toxicity. The oxidative repression of the CYP1A1 gene seems to play a central role in these regulations. NFI/CTF, which is important for the transactivation of the CYP1A1 gene promoter, is particularly sensitive to oxidative stress. A critical cysteine within the transactivating domain of NFI/CTF appears to be the target of H(2)O(2). The DNA-binding domains of several transcription factors have been described as targets of oxidative stress. However, according to Barouki and Morel (2001), recent studies suggest that more attention should be given to transactivating domains that may represent biologically relevant redox targets of cellular signaling. Thus, through the redox regulation of its transactivating function, NFI/CTF-1 constitutes a novel biologically relevant negative sensor of several stresses and therefore underscores the potential significance of the coordinated upregulation of CTF/NFI by CR in postmitotic tissues.

SIAH 2 (Z19581). This gene is a homolog of a gene studied in Drosophila photoreceptor development, which has illustrated the means by which signal transduction events regulate cell fate decisions. Development of the R7 photoreceptor is best understood and its formation is dependent on the seven in absentia (sina) gene. Hu, et al., (1997) characterized two highly conserved human homologs of sina, termed SIAH1 and SIAH2. SIAH2 maps to chromosome 3q25 and encodes a 324-amino-acid protein that shares 68% identity with Drosophila. SIAH2 was expressed in many normal and neoplastic tissues. Evidence was provided for a role in specifying cell fate and activation in apoptotic cells.

Islet mitochondrial antigen, 38 kD; imogen 44 (Z46966) encodes a mitochondrial antigen of unknown function.

F. Twenty-Five Genes Upregulated in the Four Post-Mitotic Tissues Examined (Gastrocnemius, Heart, Cerebellum and Neocortex).

Hypoxia inducible factor 1, alpha subunit (AF003695). The heterodimeric hypoxia-inducible transcription factor hif-1 is involved in the oxygen-regulated transcription of several genes including erythropoietin cloning and sequencing of the alpha-subunit of mouse (Wenger, et al., 1996). hif-1 cDNA revealed a 90% overall homology to human hif-l alpha but lack of any similarity in the 5′ untranslated region and translational start site. Mouse hif-1 alpha is encoded by an evolutionary conserved single-copy gene located on chromosome 12. Lowered expression of hif-1 in calorie restricted mice suggests better tissue oxygenation.

Importin alpha-3 subunit (AF020772) binds specifically and directly to substrates containing either a simple or bipartite nls motif and promotes docking of import substrates to the nuclear pore complex (npc). The complex is subsequently translocated through the pore by an energy requiring, ran-dependent mechanism. At the nucleoplasmic side of the npc, the three components separate and importin-alpha and -beta are re-exported from the nucleus to the cytoplasm. It is detected in all tissues examined (Ehrlich ascites tumor cells, testis, kidney, spleen, liver, heart, lung, thymus, skeletal muscle, cerebellum and brain) (Tsuji, et al., 1997).

Unknown (C76063). No known homology in GenBank.

Unknown (C79663). No known homology in GenBank.

Developmentally regulated GTP-binding protein 1 (D10715). DRG encodes a novel 41 kilodalton GTP-binding protein (DRG), which is highly expressed in the embryonic CNS and shows remarkable evolutionary conservation (Kumar, et al., 1993). Northern blots, whole-mount in situ hybridization and RNA-PCR revealed the presence of varying levels of transcript for this gene in embryos and adult tissues. Among the three mRNA species detected by northern hybridization, two smaller ones show temporally regulated expression patterns during embryonic development. Both the human and the mouse genome possess two closely related DRG genes, termed DRG1 and DRG2 (Li, et al., 2000). The two genes share 62% sequence identity at the nucleotide and 58% identity at the protein level. The corresponding proteins appear to constitute a separate family within the superfamily of the GTP-binding proteins. The DRG1 and the DRG2 mRNA are widely expressed in human and mouse tissues and show a very similar distribution pattern.

Protein transport protein SEC23A (D12713). The gene encodes a protein that covers ER-derived vesicles involved in transport from the endoplasmic reticulum to the golgi apparatus (Paccaud, et al., 1996).

ADAMTS-1 (D67076). Cleaves aggrecan, a cartilage proteoglycan (Kuno, et al., 2000) and may be involved in its turnover. Has angiogenic inhibitor activity (by similarity). It is also an active metalloprotease, which may be associated with various inflammatory processes as well as development of cancer cachexia. It cleaves aggrecan at the 1691-glu-1-leu-1692 site, within the chondroitin sulfate attachment domain.

Programmed cell death 4 (D86344). This gene, also known as the MA-3 mRNA was induced in all apoptosis-inducible cell lines tested so far, including thymocytes, T-cells, B-cells and pheochromocytoma (Shibahara, et al., 1995). The nucleotide sequence of the MA-3 cDNA predicted an amino acid (aa) sequence of 469 aa, which did not reveal significant similarity to any known proteins and functional aa motifs in databases. The MA-3 mRNA was strongly expressed in the thymus, although small amounts of the MA-3 mRNA were ubiquitously expressed in mouse adult tissues. The MA-3 gene was highly conserved during evolution and cross-hybridization bands were found not only in vertebrates but also in Drosophila melanogaster. The reduced expression of these genes induced by CR suggests lower activation of cell death programs.

Diamine acetyltransferase (spermidine/spermine N1-acetyltransferase) (SSAT) (putrescine acetyltransferase) (L10244) encodes the rate-limiting enzyme in the catabolism of polyamines. It is the key enzyme in the interconversion pathway, which leads to the formation of spermidine and putrescine from spermine and spermidine, respectively. It is also involved in the regulation of polyamine transport out of cells and, based on both functions, is importantly involved in controlling the intracellular concentration of polyamines. This is a highly regulated enzyme. This gene is induced by ischemia in the brain (Zoli, et al., 1996). Our data indicate that this is a highly expressed gene in all of the tissues studied.

Calnexin (L18888) is a calcium-binding protein that interacts with newly synthesized glycoproteins in the endoplasmic reticulum and may be involved in protein assembly. It is a molecular chaperone which may play a role in the quality control apparatus of the ER by retention of incorrectly folded proteins (Williams, 1995).

Hepatocyte nuclear factor-1-alpha (M57966) is required for the expression of several liver specific genes. It binds to the inverted palindrome 5′-gttaatnattaac-3′. The ALA-98/val-98 polymorphism is associated with a reduction in glucose-induced serum C-peptide and insulin responses and defects in the gene are a cause of maturity onset diabetes of the young type III (Ellard, 2000). The downregulation of this gene by caloric restriction may be related to insulin responses.

Butyrate response factor 2/TIS11d (M58564). This gene is a homolog of the TIS11 primary response gene that is rapidly and transiently induced by both 12-O-tetradecanoylphorbol-13-acetate and growth factors (Varnum, et al., 1991).

Tumor necrosis factor alpha-induced protein 3 (U19463) functions as an inhibitor of programmed cell death (Tewari, et al., 1995) and is found in most tissues during development. Strikingly high levels are found in lymphoid organs, including the thymus, spleen, and gut-associated lymphoid tissue. Constitutively expressed in immature and mature thymocyte subpopulations as well as in resting peripheral T-cells; activation of these leads to a down-regulation of A20. Therefore, reduced A20 levels in CR mice may be due to reduced immune and/or autoimmune activation.

Serine protease inhibitor 3 (U25844). Forms complexes with proteinases such as thrombin, trypsin, alpha-chymotrypsin, and 7S nerve growth factor (NGF), but not with urokinase or plasmin. These results, together with the immunohistochemical localization of B-43 in astrocytes and in some neurons which was observed in a previous study, suggest that B-43 may be involved in the regulation of serine proteinases present in the brain or extravasated from the blood (Nakaya, et al., 1996).

Extendin (U27830). Murine homologue of the stress-inducible phosphoprotein STI1 (also known as IEF SSP 3521 or p60). Two heat shock proteins bind to murine STI1 (mSTI1), HSC 70 and HSP 84/86 (Lassie, et al., 1997). Heat treatment caused a strong induction of mSTI1 message without affecting the steady-state level of the protein significantly. In addition, heat treatment led to changes in the isoform-composition of mSTI1. These findings suggest that the gene is involved in a stress response pathway. Lower expression in calorie restricted mice suggests reduced steady state levels of protein damage.

EAT/MCL-1 (U35623). A murine homologue of the human McI1/EAT gene, a Bcl-2 related gene. Sequence analysis revealed that murine McI1/EAT (mMcI1/EAT) has three BcI-2 homology domains, two PEST sequences, and immediate response boxes (IRB) (Okita, et al., 1998). The presence of IRB indicates that mMcI1/EAT is an immediate-early gene. mMcI1/EAT increases dramatically with exposure to retinoic acid in murine embryonal carcinoma cell lines (F9 and PCC3) as well as embryonic stem cells, both of which are models of early embryogenesis.

ABC transporter 7 protein (U43892). A novel member of the family of the ATP-binding cassette (ABC) transporters, ABC7 is conserved in mouse and in humans (Savary, et al., 1997). The ABC7 gene encodes a protein with the typical features of half-transporters, such as those involved in translocation of antigenic peptides or in peroxisomal disorders. ABC7 shows a ubiquitous expression pattern and maps to the X chromosome both in mouse and in humans. The high sequence similarity to those of two yeast half-transporters supports, once again, the extreme evolutionary conservation of this family of proteins. As shown by immunostaining using a specific antibody, the human ABC7 protein (hABC7) is a constituent of mitochondria (Csere, et al., 1998). The N-terminus of hABC7 contains the information for targeting and import into the organelles. When synthesized in yeast cells defective in Atm1p (strain delta atm1/hABC7), hABC7 protein can revert the strong growth defect observed for delta atm1 cells to near wild-type behavior. The known phenotypical consequences of inactivation of the ATM1 gene are almost fully amended by expression of hABC7 protein.

APC-binding protein EB2 (U51204). This gene was identified in a yeast two-hybrid system to search for proteins that associate with the carboxyl region of APC (Nakagawa, et al., 2000).

Chromaffin granule ATPase IA (U75321). The appearance of phosphatidylserine on the surface of animal cells triggers phagocytosis and blood coagulation. Normally, phosphatidylserine is confined to the inner leaflet of the plasma membrane by an aminophospholipid translocase, which has now been cloned and sequenced (Tang, et al., 1996). This gene is a member of a previously unrecognized subfamily of P-type adenosine triphosphatases (ATPases) that may have diverged from the primordial enzyme before the separation of the known families of ion-translocating ATPases. Studies in Saccharomyces cerevisiae suggest that aminophospholipid translocation is a general function of members of this family.

Cholinephosphate cytidylyltransferase A (phosphorvlcholine transferase A) (CTP:phosphocholine cytidylyltransferase A) (CT A) (CCT A) (CCT-ALPHA) (X54424) controls phosphatidylcholine synthesis. It catalyzes CTP+choline phosphate→pyrophosphate+CDP-choline.

H2-D (X52914). MHC I allele involved in T-cell activation (Nakamura, et al., 2000).

Gamma-Adaptin (X54424) is a subunit of the golgi adaptor. Intracellular protein transport and sorting by vesicles in the secretory and endocytic pathways requires the formation of a protein coat on the membrane. The heterotetrameric adaptor protein complex 1 (AP-1) promotes the formation of clathrin-coated vesicles at the trans-Golgi network. AP-1 interacts with various sorting signals in the cytoplasmic tails of cargo molecules, thus indicating a function in protein sorting. Mice totally deficient in gamma-adaptin die as early embryos while heterozygous knockout mice weigh less then their wild-type littermates and show impaired T-cell development (Zizioli, et al., 1999).

ATP-binding cassette subfamily A/ABC1 (X75926). The family of ATP binding cassette (ABC) transporters or traffic ATPases is composed of several membrane-associated proteins that transport a great variety of solutes across cellular membranes (Brocaddo, et al., 1999). Mutations in the gene encoding ATP-binding cassette transporter 1 (ABC1) have been reported in Tangier disease (TD), an autosomal recessive disorder that is characterized by almost complete absence of plasma high-density lipoprotein (HDL), deposition of cholesteryl esters in the reticulo-endothelial system (RES) and aberrant cellular lipid trafficking (Orso, et al., 2000). ABC1 is expressed on the plasma membrane and the Golgi complex, mediates apo-AI associated export of cholesterol and phospholipids from the cell, and is regulated by cholesterol flux. Structural and functional abnormalities in caveolar processing and the trans-Golgi secretory pathway of cells lacking functional ABC1 indicate that lipid export processes involving vesicular budding between the Golgi and the plasma membrane are severely disturbed.

S100-calcium binding protein A13 (X99921). The S100A13 cDNA codes for a novel calcium-binding protein belonging to the S100 protein family (Wicki, et al., 1996). The predicted S100A13 protein shows sequence homologies to other S10O proteins between 50.5% (to S100A5) and 59.3% (to S100A12). High mRNA amounts were reported in skeletal muscle, heart, kidney, ovary, small intestine and pancreas. Similar to the putative human protein, mouse S100A13 is composed of 98 amino acids displaying a homology of 86.7% compared to human S100A13.

Cyclin A/CDK-2-Associated Protein P19 (Z47088) is involved in RNA Polymerase elongation and also functions as a transcriptional factor. It interacts with the cyclin A/CDK-2 complex.

### REFERENCES

1. Bachiller D, Klingensmith J, Kemp C, Belo J A, Anderson R M, May S R, McMahon J A, McMahon A P, Harland R M, Rossant J, De Robertis E M. The organizer factors Chordin and Noggin are required for mouse forebrain development. Nature 403:658-61, 2000.

2. Bao S, Zhu J, Garvey W T. Cloning of Rab GTPases expressed in human skeletal muscle: studies in insulin-resistant subjects. Horm. Metab. Res. 30:656-62, 1998.

3. Barouki R, Morel Y. Repression of cytochrome P450 1A1 gene expression by oxidative stress: mechanisms and biological implications. Biochem. Pharmacol. 61:511-6, 2001.

4. Beckman K B, Ames B N: The free radical theory of aging matures. Physiol. Revs. 78:547-81,1998.

5. Bellomo D, Headrick J P, Silins G U, Paterson C A, Thomas P S, Gartside M, Mould A, Cahill M M, Tonks I D, Grimmond S M, Townson S, Wells C, Little M, Cummings M C, Hayward N K, Kay G F. Mice lacking the vascular endothelial growth factor-B gene (Vegfb) have smaller hearts, dysfunctional coronary vasculature, and impaired recovery from cardiac ischemia. Circ. Res. 86:E29-35, 2000.

6. Bottero V, Rossi F, Samson M, Mari M, Hofman P, Peyron J F. IkappaB Alpha, the NF-kappaB inhibitory subunit, interacts with ANT, the mitochondrial ATP/ADP translocator. J. Biol. Chem. 2001 Apr 3; [epub ahead of print].

7. Brilliant M H. The mouse p (pink-eyed dilution) and human P genes, oculocutaneous albinism type 2 (OCA2), and melanosomal pH. Pigment Cell Res. 14:86-93, 2000.

8. Broccardo C, Luciani M, Chimini G. The ABCA subclass of mammalian transporters. Biochim. Biophys. Acta 1461:395-404, 1999.

9. Bruce-Keller A J, Umberger G, McFall R, Mattson M P. Food restriction reduces brain damage and improves behavioral outcome following excitotoxic and metabolic insults. Ann. Neurol. 45:8-15,1999.

10. Chen F W, Ioannou Y A. Ribosomal proteins in cell proliferation and apoptosis. Int. Rev. Immunol. 18:429-48,1999.

11. Csere P, Lill R, Kispal G. Identification of a human mitochondrial ABC transporter, the functional orthologue of yeast Atm1 p. FEBS Lett. 441:266-70, 1998.

12. Cwikla S J, Tsuji T, McBride J, Wong D T, Todd R. doc-1—mediated apoptosis in malignant hamster oral keratinocytes. J. Oral Maxillofac. Surg. 58:406-14, 2000.

13. Dormady S P, Zhang X M, Basch R S. Hematopoietic progenitor cells grow on 3T3 fibroblast monolayers that overexpress growth arrest-specific gene-6 (GAS6). Proc. Natl. Acad. Sci. USA 97:12260-5, 2000.

14. Duan W, Mattson M P. Dietary restriction and 2-deoxyglucose administration improve behavioral outcome and reduce degeneration of dopaminergic neurons in models of Parkinson's disease. J. Neurosci. Res. 57:195-206, 1999.

15. Durrington P N, Mackness B, Mackness M I. Paraoxonase and atherosclerosis. Arterioscler. Thromb. Vasc. Biol. 21:473-80, 2001.

16. Ellard S. Hepatocyte nuclear factor 1 alpha (HNF-1 alpha) mutations in maturity-onset diabetes of the young. Hum. Mutat. 16:377-85, 2000.

17. Elzinga S D, van Oosterum K, Maat C, Grivell L A, van der Spek H. Isolation and RNA-binding analysis of NAD+-isocitrate dehydrogenases from Kluyveromyces lactis and Schizosaccharomyces pombe. Curr. Genet. 38:87-94, 2000.

18. Fishbein L: Biological effects of dietary restriction. New York: Springer-Verlag; 1991.

19. Forrest G L, Gonzalez B, Tseng W, Li X, Mann J. Human carbonyl reductase overexpression in the heart advances the development of doxorubicin-induced cardiotoxicity in transgenic mice. Cancer Res. 60:5158-64, 2000.

20. Frame L T, Hart R W, Leakey J E. Caloric restriction as a mechanism mediating resistance to environmental disease. Envir. Health Perspect. 106 Suppl. 1:313-24,1998.

21. Fujita A, Kurachi Y. SAP family proteins. Biochem. Biophys. Res. Commun. 269:1-6, 2000.

22. Goalstone M L and Draznin B. Effect of insulin on farnesyltransferase activity in 3T3-L1 adipocytes. J. Biol. Chem. 271:27585-9,1996.

23. Goalstone M L, Leitner J W, Berhanu P, Sharma P M, Olefsky J M, Draznin B. Insulin signals to prenyltransferases via the Shc branch of intracellular signaling. J. Biol. Chem. 276:12805-12, 2001.

24. Gong Y, Krakow D, Marcelino J, Wilkin D, Chitayat D, Babul-Hirji R, Hudgins L, Cremers C W, Cremers F P, Brunner H G, Reinker K, Rimoin D L, Cohn D H, Goodman F R, Reardon W, Patton M, Francomano C A, Warman M L. Heterozygous mutations in the gene encoding noggin affect human joint morphogenesis. Nat. Genet. 21:302-4, 1999.

25. Gonzalez-Amaro R, Sanchez-Madrid F. Cell adhesion molecules: selectins and integrins. Crit. Rev. Immunol. 19:389-429, 1999.

26. Grinberg A V, Hannemann F, Schiffler B, Muller J, Heinemann U, Bernhardt R. Adrenodoxin: structure, stability, and electron transfer properties. Proteins 40:590-612, 2000.

27. Hendrie H C, Ogunniyi A, Hall K S, Baiyewu O, Unverzagt F W, Gureje O, Gao S, Evans R M, Ogunseyinde A O, Adeyinka A O, Musick B, Hui S L. Incidence of dementia and Alzheimer disease in 2 communities: Yoruba residing in Ibadan, Nigeria, and African Americans residing in Indianapolis, Ind. JAMA 285:739-47, 2001.

28. Hengstschlager M, Rodman D M, Miloloza A, Hengstschlater-Ottnad E, osner M, Kubista M. Tuberous sclerosis gene products in proliferation control. Mutat. Res. 488:233-239, 2001.

29. Hishikawa K, Nakaki T, Fujii T. Transforming growth factor-beta(1) induces apoptosis via connective tissue growth factor in human aortic smooth muscle cells. Eur. J. Pharmacol. 385:287-90, 1999.

30. Honda Y, Honda S. The daf-2 gene network for longevity regulates oxidative stress resistance and Mn-superoxide dismutase gene expression in Caenorhabditis elegans. FASEB J. 13:1385-93,1999.

31. Hu G, Chung Y L, Glover T, Valentine V, Look A T, Fearon E R. Characterization of human homologs of the Drosophila seven in absentia (sina) gene. Genomics 46:103-11, 1997.

32. Huang J, Kim L J, Mealey R, Marsh H C J, Zhang Y, Tenner A J, Connolly E S J, Pinsky D J: Neuronal protection in stroke by an sLex-glycosylated complement inhibitory protein. Science 285:595-599, 1999.

33. Jazwinski S M: Longevity, genes, and aging. Science 273:54-9, 1996.

34. Johnson F B, Sinclair D A, Guarente L: Molecular biology of aging. Cell 96:291-302, 1999.

35. Johnson T E: Increased life-span of age-1 mutants in Caenorhabditis elegans and lower Gompertz rate of aging. Science 249:908-12, 1990.

36. Kanoh N, Dai C F, Tanaka T, Izawa D, Li Y F, Kawashima H, Miyasaka M. Constitutive expression of GlyCAM-1 core protein in the rat cochlea. Cell Adhes. Commun. 7:259-66,1999.

37. Kim K W, Chung H H, Chung C W, Kim I K, Miura M, Wang S, Zhu H, Moon K D, Rha G B, Park J H, Jo D G, Woo H N, Song Y H, Kim B J, Yuan J, Jung Y K. Inactivation of farnesyltransferase and geranylgeranyltransferase I by caspase-3: Cleavage of the common alpha subunit during apoptosis. Oncogene 20:358-66, 2001.

38. Kipreos E T, Pagano M. The F-box protein family. Genome Biol. 1:Reviews 3002, 2000.

39. Klein M G, Yao Y, Slosberg E D, Lima C D, Doki Y, Weinstein I B. Characterization of PKCI and comparative studies with FHIT, related members of the HIT protein family. Exp. Cell Res. 244:26-32, 1998.

40. Klesert T R, Cho D H, Clark J I, Maylie J, Adelman J, Snider L, Yuen E C, Soriano P, Tapscott S J. Mice deficient in Six5 develop cataracts: implications for myotonic dystrophy. Nat. Genet. 25:105-9, 2000.

41. Kohrl J, Brigelius-Flohe R, Bock A, Gartner R, Meyer O, Flohe L. Selenium in biology: facts and medical perspectives. Biol. Chem. 381:849-64, 2000.

42. Kumar S, Iwao M, Yamagishi T, Noda M, Asashima M. Expression of GTP-binding protein gene drg during Xenopus laevis development. Int. J. Dev. Biol. 37:539-46,1993.

43. Kuno K, Okada Y, Kawashima H, Nakamura H, Miyasaka M, Ohno H, Matsushima K. ADAMTS-1 cleaves a cartilage proteoglycan, aggrecan. FEBS Lett. 478:241-5, 2000.

44. Lassle M, Blatch G L, Kundra V, Takatori T, Zetter B R. Stress-inducible, murine protein mSTI1. Characterization of binding domains for heat shock proteins and in vitro phosphorylation by different kinases. J. Biol. Chem. 272:1876-84,1997.

45. Lee C-K, Weindruch R, Prolla T A: Gene expression profile of the aging brain. Nat. Genet. 25:294-297, 2000.

46. Lee S J, Benveniste E N. Adhesion molecule expression and regulation on cells of the central nervous system. J. Neuroimmunol. 98:77-88,1999.

47. Li B, Trueb B. DRG represents a family of two closely related GTP-binding proteins. Biochim. Biophys. Acta 1491:196-204, 2000.

48. Li X, Eriksson U. Novel VEGF family members: VEGF-B, VEGF-C and VEGF-D. Int. J. Biochem. Cell Biol. 33:421-6, 2001.

49. Lin Y J, Seroude L, Benzer S: Extended life-span and stress resistance in the drosophila mutant methuselah. Science 282:943-946,1998.

50. Lockhart D J, Dong H L, Byrne M C, Follettie M T, Gallo M V, Chee M S, Mittmann M, Wang C W, Kobayashi M, Horton H, Brown E L: Expression monitoring by hybridization to high-density oligonucleotide arrays. Nat. Biotechnol. 14:1675-80, 1996.

51. Logroscino G, Marder K, Cote L, Tang M X, hea S, Mayeux R. Dietary lipids and antioxidants in Parkinson's disease: a population-based, case-control study. Ann. Neurol. 39:89-94,1996.

52. Lombard D B, Guarente L: Cloning the gene for Werner syndrome: a disease with many symptoms of premature aging. Trends in Genetics 12:283-6, 1996.

53. Martin G M, Austad S N, Johnson T E: Genetic analysis of aging: Role of oxidative damage and environmental stresses. Nat. Genet. 13:25-34,1996.

54. Masoro E J. Influence of caloric intake on aging and on the response to stressors. J. Toxicol. Envir. Health (Part B) 1:243-57, 1998.

55. Mayeux R, Costa R, Bell K, Merchant C, Tang M-X, Jacobs D. Reduced risk of Alzheimer's disease among individuals with low caloric intake. Neurology 52:A296-7, 1999.

56. McCarter R J. Role of caloric restriction in the prolongation of life. Clin. Geriatr. Med. 11:553-65, 1995.

57. Melaragno M G, Fridell Y W, Berk BC. The Gas6/AxI system: a novel regulator of vascular cell function. Trends Cardiovasc. Med. 9:250-3, 1999.

58. Miura M, Hatakeyama S, Hattori K, Nakayama K. Structure and expression of the gene encoding mouse F-box protein, Fwd2. Genomics 62:50-8,1999.

59. Morel Y, Coumoul X, Nalpas A, Barouki R. Nuclear factor I/CCMT box transcription factor trans-activating domain is a negative sensor of cellular stress. Mol. Pharmacol. 58:1239-46, 2000.

60. Moussad E E, Brigstock D R. Connective tissue growth factor: what's in a name? Mol. Genet. Metab. 71:276-92, 2000.

61. Murakami S, Johnson T E: A genetic pathway conferring life extension and resistance to UV stress in Caenorhabditis elegans. Genetics 143:1207-18, 1996.

62. Muzio M, Polentarutti N, Bosisio D, Manoj Kumar P P, Mantovani A. Toll-like receptor family and signalling pathway. Biochem. Soc. Trans. 28:563-6, 2000.

63. Nakagawa H, Koyama K, Murata Y, Morito M, Akiyama T, Nakamura Y. EB3, a novel member of the EB1 family preferentially expressed in the central nervous system, binds to a CNS-specific APC homologue. Oncogene 19:210-6, 2000.

64. Nakagawa T, Yuan J. Cross-talk between two cysteine protease families. Activation of caspase-12 by calpainin apoptosis. J. Cell Biol. 150:887-94, 2000.

65. Nakamura M C, Hayashi S, Niemi E C, Ryan J C, Seaman W E. Activating Ly-49D and inhibitory Ly-49A natural killer cell receptors demonstrate distinct requirements for interaction with H2-D(d). J. Exp. Med. 192:447-54, 2000.

66. Nakaya N, Nishibori M, Kawabata M, Saeki K. Cloning of a serine proteinase inhibitor from bovine brain: expression in the brain and characterization of its target proteinases. Brain Res. Mol. Brain Res. 42:293-300,1996.

67. Ogg S, Paradis S, Gottlieb S, Patterson G I, Lee L, Tissenbaum H A, Ruvkun G. The Fork head transcription factor DAF-16 transduces insulin-like metabolic and longevity signals in C. elegans. Nature 389:994-9,1997.

68. Okita H, Umezawa A, Suzuki A, Hata J. Up-regulated expression of murine McI1/EAT, a bcI-2 related gene, in the early stage of differentiation of murine embryonal carcinoma cells and embryonic stem cells. Biochim. Biophys. Acta 1398:335-41, 1998.

69. Orr W C, Sohal R S: Extension of life-span by over-expression of superoxide dismutase and catalase in Drosophila melanogaster. Science 263:1128-1130, 1994.

70. Orso E, Broccardo C, Kaminski W E, Bottcher A, Liebisch G, Drobnik W, Gotz A, Chambenoit O, Diederich W, Langmann T, Spruss T, Luciani M F, Rothe G, Lackner K J, Chimini G, Schmitz G. Transport of lipids from golgi to plasma membrane is defective in tangier disease patients and Abc1-deficient mice. Nat. Genet. 24:192-6, 2000.

71. Osada T, Ikegami S, Takiguchi-Hayashi K, Yamazaki Y, Katoh-Fukui Y, Higashinakagawa T, Sakaki Y, Takeuchi T. Increased anxiety and impaired pain response in puromycin-sensitive aminopeptidase gene-deficient mice obtained by a mouse gene-trap method. J. Neurosci. 19(14):6068-78, 1999.

72. Paccaud J P, Reith W, Carpentier J L, Ravazzola M, Amherdt M, Schekman R, Orci L. Cloning and functional characterization of mammalian homologues of the COPII component Sec23. Mol. Biol. Cell. 7:1535-46, 1996.

73. Paradis S, Ruvkun G: Caenorhabditis elegans Akt/PKB transduces insulin receptor-like signals from AGE-1 P13 kinase to the DAF-16 transcription factor. Genes Dev. 12:2488-98,1998.

74. Parfett C L, Pilon R. Oxidative stress-regulated gene expression and promotion of morphological transformation induced in C3H/10T1/2 cells by ammonium metavanadate. Food Chem. Toxicol. 33:301-8,1995.

75. Parkes T L, Elia A J, Dickinson D, Hilliker A J, Phillips J P, Boulianne G L: Extension of Drosophila lifespan by overexpression of human SOD1 in motorneurons [comment] [see comments]. Nat. Genet. 19:171-4,1998.

76. Pasinetti G M, Hassler M, Stone D, Finch C E: Glial gene expression during aging in rat striatum and in long-term responses to 6-OHDA lesions. Synapse 31:278-84, 1999.

77. Paule M R, White R J. Survey and summary: transcription by RNA polymerases I and III. Nucleic Acids Res. 28:1283-98, 2000.

78. Peters L L, John K M, Lu F M, Eicher E M, Higgins A, Yialamas M, Turtzo L C, Otsuka A J, Lux S E. J. Cell Biol. 130:313-330, 1995.

79. Pomposiello P J, Demple B. Redox-operated genetic switches: the SoxR and OxyR transcription factors. Trends Biotechnol. 19:109-14, 2001.

80. Pugh T D, Klopp R G, Weindruch R: Controlling caloric consumption: Protocols for rodents and rhesus monkeys. Neurobiol. Aging 20:157-165, 1999.

81. Rattan S I. Synthesis, modifications, and turnover of proteins during aging. Exp. Gerontol. 31:33-47, 1996.

82. Rubtsov A M, Lopina O D. Ankyrins. FEBS Lett. 482:1-5, 2000.

83. Sanchez J C, Schaller D, Ravier F, Golaz O, Jaccoud S, Belet M, Wilkins M R, James R, Deshusses J, Hochstrasser D. Translationally controlled tumor protein: a protein identified in several nontumoral cells including erythrocytes. Electrophoresis 18:150-5, 1997.

84. Savary S, Allikmets R, Denizot F, Luciani M F, Mattei M G, Dean M, Chimini G. Isolation and chromosomal mapping of a novel ATP-binding cassette transporter conserved in mouse and human. Genomics 41:275-8,1997.

85. Shibahara K, Asano M, Ishida Y, Aoki T, Koike T, Honjo T. Isolation of a novel mouse gene MA-3 that is induced upon programmed cell death. Gene 166:297-301, 1995.

86. Shikama N, Brack C. Changes in the expression of genes involved in protein synthesis during Drosophila aging. Gerontology 142:123-36, 1996.

87. Sohal R S, Weindruch R. Oxidative stress, caloric restriction, and aging. Science 273:59-63,1996.

88. Soltys B J, Dongchon K, Gupta R S. Localization of P32 protein (gC1q-R) in mitochondria and at specific extramitochondrial locations in normal tissues. Histochem. Cell Biol. 114:245-55, 2000.

89. Steingrimsson E, Sawadogo M, Gilbert D J, Zervos A S, Brent R, Blanar M A, Fisher D E, Copeland N G, Jenkins N A. Murine chromosomal location of five bHLH-Zip transcription factor genes. Genomics 28:179-83, 1995.

90. Sumitomo K, Kurisaki A, Yamakawa N, Tsuchida K, Shimizu E, Sone S, Sugino H. Expression of a TGF-betal inducible gene, TSC-36, causes growth inhibition in human lung cancer cell lines. Cancer Lett. 155:37-46, 2000.

91. Tang X, Halleck M S, Schlegel R A, Williamson P. A subfamily of P-type ATPases with aminophospholipid transporting activity. Science 272:1495-7, 1996.

92. Tewari M, Wolf F W, Seldin M F, O'Shea K S, Dixit V M, Turka L A. Lymphoid expression and regulation of A20, an inhibitor of programmed cell death. J. Immunol. 154:1699-706, 1995.

93. Tissenbaum H A, Ruvkun G: An insulin-like signaling pathway affects both longevity and reproduction in Caenorhabditis elegans. Genetics 148:703-17, 1998.

94. Todd R, McBride J, Tsuji T, Donoff R B, Nagai M, Chou M Y, Chiang T, Wong D T. Deleted in oral cancer-1 (doc-1), a novel oral tumor suppressor gene. FASEB J. 9:1362-70, 1995.

95. Tsaioun K I. Vitamin K-dependent proteins in the developing and aging nervous system. Nutr. Rev. 57:231-40, 1999.

96. Tsuji, L., Takumi, T., Imamoto, N. and Yoneda, Y. Identification of novel homologues of mouse importin alpha, the alpha subunit of the nuclear pore-targeting complex, and their tissue-specific expression. FEBS Lett. 416:30-34, 1997.

97. Varnum B C, Ma Q F, Chi T H, Fletcher B, Herschman H R. The TIS11 primary response gene is a member of a gene family that encodes proteins with a highly conserved sequence containing an unusual Cys-His repeat. Mol. Cell. Biol. 11:1754-8,1991.

98. Weindruch R, Walford R L. The Retardation of Aging and Disease by Dietary Restriction. Thomas, Springfield, Ill., 1988.

99. Wenger R H, Rolfs A, Marti H H, Guenet J L, Gassmann M. Nucleotide sequence, chromosomal assignment and mRNA expression of mouse hypoxia-inducible factor-1 alpha. Biochem. Biophys. Res. Commun. 223:54-9, 1996.

100. Wicki R, Schafer B W, Erne P, Heizmann C W. Characterization of the human and mouse cDNAs coding for S100A13, a new member of the S100 protein family. Biochem. Biophys. Res. Commun. 227:594-9,1996.

101. Williams D B. The Merck Frosst Award Lecture 1994/La conference Merck Frosst 1994. Calnexin: a molecular chaperone with a taste for carbohydrate. Biochem. Cell Biol. 73:123-32, 1995.

102. Wishart M J, Dixon J E. Gathering STYX: phosphatase-like form predicts functions for unique protein-interaction domains. Trends Biochem. Sci. 23:301-6,1998.

103. Xi H, Shin W S, Suzuki J, Nakajima T, Kawada T, Uehara Y, Nakazawa M, Toyo-oka T. Dystrophin disruption might be related to myocardial cell apoptosis caused by isoproterenol. J. Cardiovasc. Pharmacol. 36 Suppl. 2:S25-9, 2000.

104. Xu A, Bellamy A R, Taylor J A. Expression of translationally controlled tumour protein is regulated by calcium at both the transcriptional and post-transcriptional level. Biochem. J. 342:683-9,1999.

105. Yet S F, McA'Nulty M M, Folta S C, Yen H W, Yoshizumi M, Hsieh C M, Layne M D, Chin M T, Wang H, Perrella M A, Jain M K, Lee M E Human E Z F, a Kruppel-like zinc finger protein, is expressed in vascular endothelial cells and contains transcriptional activation and repression domains. J. Biol. Chem. 273:1026-31, 1998.

106. Yu B P: Modulation of Aging Processes by Dietary Restriction. Boca Raton, Fla.:CRC Press; 1994.

107. Yu C E, Oshima J, Fu Y H, Wijsman E M, Hisama F, Alisch R, Matthews S, Nakura J, Miki T, Ouais S, Martin G M, Mulligan J, Schellenberg G D: Positional cloning of the Werner's syndrome gene. Science 272:258-62, 1996.

108. Zizioli D, Meyer C, Guhde G, Saftig P, von Figura K, Schu P. Early embryonic death of mice deficient in gamma-adaptin. J. Biol. Chem. 274:5385-90,1999.

109. Zoli M, Pedrazzi P, Zini I, Agnati L F. Spermidine/spermine N1-acetyltransferase mRNA levels show marked and region-specific changes in the early phase after transient forebrain ischemia. Brain Res. Mol. Brain Res. 38:122-34, 1996.

We claim:

1. A method of measuring a relative metabolic state of a multicellular organism comprising the steps of:
(a) obtaining a sample from a subject;
(b) determining the gene expression pattern of at least one of the ORFs selected from the group consisting of ORFs D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088; and
(c) determining whether the gene expression profile of step (b) is more similar to a CR-induced metabolic state or a standard diet metabolic state.

2. The method of claim 1 wherein the sample comprises an organ, tissue or cell.

3. The method of claim 1 wherein said determining step comprises detecting RNA or cDNA encoded by at least one of the ORFs listed in (b).

4. The method of claim 1 wherein said determining step comprises detecting protein encoded by at least one of the ORFs listed in (b).

5. The method of claim 1 wherein the expression pattern of at least one sequence selected from the group consisting of D31966, R74626, U79163, M22531, U43285, U79523, X81059, and X84239 is determined in step (b).

6. The method of claim 1 wherein the expression pattern of at least one sequence selected from the group consisting of U84411, U51167, U43836, U60001, D49473, L08651, U28917, X59846, M 17417, AF011644, AJ01101, D16333, D49744, L29123, M74555, U19799, U20344, U35312, U43512, U47543, U56773, X54352, Z19581, AF003695, C76063, D10715, D12713, D86344, L18888, U27830, U43892, U51204, U75321, X54424, and Z47088 is determined in step (b).

7. The method of claim 1 wherein the expression patterns of at least five sequences are determined in step (b).

8. The method of claim 7 wherein the expression patterns of at least ten sequences are determined in step (b).

9. The method of claim 8 wherein the expression patterns of at least twenty sequences are determined in step (b).

10. The method of claim 1 wherein the organism is a mammal.

11. The method of claim 10 wherein the mammal is selected from the group consisting of humans, rats and mice.

12. The method of claim 2 wherein the sample is a tissue selected from the group consisting of neocortex, cerebellum, heart tissue, liver tissue, kidney and skeletal muscle.

13. A method for screening a compound for the ability to modulate the metabolic state in a multicellular organism comprising the steps of:
(a) dividing test organisms into first and second groups;
(b) exposing the organisms of the first group to a test compound;
(c) analyzing samples of the first and second groups for the gene expression pattern of at least one of the genes selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088; and
(d) comparing the analysis of the first and second groups and identifying test compounds that modify the expression of the sequences of step (c) in the first group such that the expression patterns are more similar to those observed in CR-treated animals.

14. The method of claim 13 wherein the sample comprises an organ, tissue or cell.

15. The method of claim 13 wherein said determining step comprises detecting RNA or cDNA encoded by at least one of the ORFs listed in (c).

16. The method of claim 13 wherein said determining step comprises detecting protein encoded by at least one of the ORFs listed in (c).

17. The method of claim 13 wherein the expression pattern of at least one sequence selected from the group consisting of D31966, R74626, U79163, M22531, U43285, U79523, X81059, and X84239 is determined in step (b).

18. The method of claim 13 wherein the expression pattern of at least one sequence selected from the group consisting of sequence comprises U84411, U51167, U43836, U60001, D49473, L08651, U28917, X59846, AA117417, AF011644, AJ001101, D16333, D49744, L29123, M74555, U19799, U20344, U35312, U43512, U47543, U56773, X54352, Z19581, AF003695, C76063, D10715, D12713, D86344, L18888, U27830, U43892, U51204, U75321, X54424, and Z47088 is determined in step (b).

19. The method of claim 13 wherein the expression patterns of at least five sequences are determined in step (b).

20. The method of in claim 13, wherein the organism is a mammal.

21. The method of claim 20, wherein the mammal is selected from the group consisting of humans, rats and mice.

22. The method of in claim 14, wherein the tissue is selected from the group consisting of cerebullum, neocortex, heart tissue, skeletal muscle, liver and kidney tissue.

23. A method of mimicking the CR metabolic state in an organism, comprising the step of manipulating the expression of at least one gene selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088, wherein the expression of a biomarker gene that decreases in response to CR is decreased and wherein the expression of a biomarker gene that is known to increase in response to CR is increased.

24. A method of mimicking the CR metabolic state comprising the step of using pharmaceutical compounds that either mimic, inhibit or enhance the activity of proteins encoded by at least one of the genes selected from the group consisting of ORFs D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088.

25. A method of mimicking the CR metabolic state comprising the step of using nutritional or nutraceutical compounds that mimic, enhance or inhibit the activity of proteins encoded by at least one of the genes selected from the group consisting of ORFs D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088.

26. A kit for the detection of measuring the CR metabolic state of a multicellular organism, comprising reagents suitable for quantitatively measuring protein, mRNA or cDNA levels of proteins, mRNAs or cDNAs encoded by ORFs D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088.
